(19) United States
(12) Patent Application Publication
(10) Pub. No.: US 2024/0139148 A1
(43) Pub. Date: May 2, 2024

(54) COMPOSITIONS AND METHODS FOR TREATING NON-TUBERCULOUS MYCOBACTERIAL INFECTIONS

(71) Applicant: COLORADO STATE UNIVERSITY RESEARCH FOUNDATION, Fort Collins, CO (US)

(72) Inventors: Mary Jackson, Fort Collins, CO (US); Juan Belardinelli, Fort Collins, CO (US)

(21) Appl. No.: 18/568,615

(22) PCT Filed: Jun. 10, 2022
(86) PCT No.: PCT/US22/33043

§ 371 Date: Dec. 8, 2023

Related U.S. Application Data
(60) Provisional application No. 63/276,488, filed on Nov. 5, 2021.
(60) Provisional application No. 63/209,472, filed on Jun. 11, 2021.

(51) Int. Cl. A61K 31/357; A61K 31/5377; A61P 31/08

(52) U.S. Cl. CPC A61K 31/357; A61K 31/5377; A61P 31/08

(57) ABSTRACT

The present disclosure provides compositions and methods related to the treatment of non-tuberculous mycobacterial infections in a subject. In particular, the present disclosure provides compositions comprising an anti-malarial agent and associated methods of treating and/or preventing a non-tuberculous mycobacterial infection in a subject using the composition.

### CROSS REFERENCE TO RELATED APPLICATIONS

This application claims priority to and the benefit of U.S. Provisional Patent Application No. 63/209,472 filed Jun. 11, 2021, and U.S. Provisional Patent Application No. 63/276,488 filed Nov. 5, 2021, both of which are incorporated herein by reference in their entireties for all purposes.

### GOVERNMENT FUNDING

This invention was made with government support under grant number R21 AI147326 awarded by the National Institutes of Health (NIH). The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,524 Byte ASCII (Text) file named “40834-601_SEQUENCE-LISTING_ST25” created on May 19, 2022.

### FIELD

The present disclosure provides compositions and methods related to the treatment of non-tuberculous mycobacterial infections in a subject. In particular, the present disclosure provides compositions comprising an anti-malarial agent and associated methods of treating and/or preventing a non-tuberculous mycobacterial infection in a subject using the composition.

### BACKGROUND

Rapidly-growing nontuberculous mycobacteria (NTM) of the Mycobacterium abscessus complex (MABSC; including M. abscessus subsp. abscessus, M. abscessus subsp. massiliense and M. abscessus subsp. bolletii), and slowly-growing NTM of the Mycobacterium avium complex (MAC) have emerged as important human pathogens globally, and are linked to an increasing number of pulmonary infections among patients with structural lung disease such as chronic obstructive pulmonary disease (COPD), bronchiectasis, and cystic fibrosis (CF). The low cure rate of currently available treatment regimens, in spite of a minimum of 12 months of chemotherapy (in the range of 25-58% for MABSC pulmonary disease), notable side effects, and frequent bacterial re-emergence associated with these regimens highlight the need for alternative approaches to treat NTM infections. The radiographic appearance of MAC and MABSC pulmonary disease can be similar to tuberculosis (TB) and may include cavity formation, nodules, and airspace disease. When present, granulomas from MABSC-infected patients are histologically indistinguishable from lesions caused by Mycobacterium tuberculosis (Mtb). The precise nature of the host stresses to which MABSC and MAC are exposed within these lesions and how the bacteria respond to them is not as well documented than for Mtb. Nevertheless, it is believed that NTM and Mtb share the ability to persist within the granulomatous structures in a non-replicating state, which contributes to their drug tolerance and to treatment failure in chronically infected individuals. Further compounding this problem is the ability of MABSC and MAC to form what appears to be genetically programmed biofilms when in contact with human cells, and clinical evidence for MABSC biofilm formation within the thickened alveolar walls, airways, and lung cavity during pulmonary infections in patients with COPD or CF. Biofilm formation is a common strategy used by pathogens of the soft tissue and airways to chronically infect the host and can enhance their drug tolerance and resistance to host defense mechanisms.

The inhibition of aerobic respiration caused by oxygen depletion or exposure to nitric oxide (NO) and carbon monoxide is a common stressor of intra- and extracellular pathogenic mycobacteria inside activated immune cells, in avascular necrotic regions of granulomas, and within microaggregates or biofilms. Mtb survives this stress in vitro and in vivo by inducing a regulon of 48 genes that drives its entry into a non-replicating state while adapting bacterial metabolism to maintain energy levels and a redox balance compatible with survival in the absence of respiration. This response is under control of the three-component regulatory system, DosRST, and inhibitors of the response regulator DosR and of the two sensor histidine kinases. DosS and DosT, are being sought for their potential to shorten tuberculosis treatment and lower relapse rates when used in combination with standard-of-care antibiotics.

MABSC species are endowed with a DosRS system, although they lack the sensor histidine kinase, DosT. The precise nature of the MABSC DosRS regulon has yet to be experimentally established, but a recent study identified the predicted DosR regulon genes of MABSC among genes whose expression was the most upregulated following exposure to NO-induced hypoxia. Further supporting a role for this two-component regulatory system (2CR) in the ability of MABSC to become a chronic pathogen of the lung, dosR, dosS and other predicted DosR-regulated genes were found to be expressed at higher levels in rough (R) than in smooth (S) morphotype variants of M. abscessus subsp. abscessus (Mabs) ATCC 19977, the former variants being known for their hyperaggregative and hypervirulent phenotypes. In view of these findings, a detailed investigation of DosRS from MABSC was undertaken herein with the goal to define the contribution of this 2CR to MABSC drug tolerance and ability to survive under microaerophilic conditions, including within biofilms. Genetic and chemical inhibition of DosRS in vitro and in vivo validate this regulator as a target of therapeutic interest whose inhibition has the potential to improve MABSC clearance and treatment outcomes.

### SUMMARY

Embodiments of the present disclosure include a composition for treating a non-tuberculous mycobacterial infection in a subject. In accordance with these embodiments, the composition includes a therapeutically effective dose of at least one anti-malarial agent, and a pharmaceutically acceptable carrier.

In some embodiments, the non-tuberculous mycobacterial infection is caused by a non-tuberculous mycobacteria (NTM) selected from the group consisting of: M. abscessus subsp. abscessus, M. abscessus subsp. massiliense, M. abscessus subsp. bolletii, Mycobacterium avium complex. M. kansasii, M. chelonae, M. fortuitum, and M. xenopi, M. peregrinum, M. mucogenicum, M. senegalense, M. immunogenum, M. simiae, M. gordonae, M. septicum, M. malmoense, M. goodii, M. haemophilum, M. genavense, M. scrofulaceum, M. lentiflavum, M. bohemicum, and M. marinum.

In some embodiments, the non-tuberculous mycobacterial infection is caused by a mycobacterium selected from the group consisting of: M. leprae, and M. ulcerans

In some embodiments, the composition treats and/or prevents at least one symptom of the non-tuberculous mycobacterial infection in the subject. In some embodiments, the symptom is selected from the group consisting of a cough, weight loss, appetite loss, fatigue, shortness of breath, a fever, and night sweats.

In some embodiments, the at least one anti-malarial agent comprises a trioxolane (secondary ozonide). In some embodiments, the at least one anti-malarial agent comprises a synthetic peroxide. In some embodiments, the at least one anti-malarial agent comprises arterolane (OZ277). In some embodiments, the at least one anti-malarial agent consists essentially of arterolane (OZ277). In some embodiments, the at least one anti-malarial agent comprises ozonide OZ439. In some embodiments, the at least one anti-malarial agent consists essentially of ozonide OZ439. In some embodiments, the at least one anti-malarial agent comprises OZ277 and OZ439. In some embodiments, the at least one anti-malarial agent consists essentially of OZ277 and OZ439.

In some embodiments, the at least one anti-malarial agent is present in the composition in an amount ranging from about 5 mg to about 1200 mg. In some embodiments, the OZ277 is present in the composition in an amount ranging from about 5 mg to about 1200 mg. In some embodiments, the OZ439 is present in the composition in an amount ranging from about 5 mg to about 1200 mg.

In some embodiments, the composition further comprises at least one antibiotic. In some embodiments, the at least one antibiotic is selected from the group consisting of: clarithromycin, azithromycin, rifampin, rifabutin, ethambutol, streptomycin, amikacin, ciprofloxacin, doxycycline, clofazimine, isoniazid, linezolid, moxifloxacin, trimethoprim/sulfamethoxazole, cefoxitin, imipenem, and tigecycline.

In some embodiments, composition does not comprise an antibiotic.

Embodiments of the present disclosure also include a method of treating and/or preventing a non-tuberculous mycobacterial infection in a subject. In accordance with these embodiments, the method includes administering any of the compositions described herein to the subject.

In some embodiments, the composition is administered in a single dose. In some embodiments, the composition is administered in multiple doses. In some embodiments, the composition is administered daily, weekly, bi-weekly or monthly.

In some embodiments, the composition is formulated as a tablet, capsule, powder, granule, sachet, gel, liquid, spray, suspension, or inhalant. In some embodiments, the composition is administered to the subject orally, sublingually, topically, intravenously, subcutaneously, transcutaneously, nasally, vaginally, rectally, or by inhalation. In some embodiments, the composition is administered to the subject at a dose ranging from about 50 mg/kg to about 1200 mg/kg. In some embodiments, the composition is administered to the subject in an amount ranging from about 50 mg/dose to about 1200 mg/dose.

In some embodiments, the composition is administered with an antibiotic. In some embodiments, the composition is administered with the antibiotic in the same dose or in a different dose. In some embodiments, the composition is not administered with an antibiotic.

In some embodiments, administration of the composition enhances at least one of oxidative stress, apoptosis, autophagy and/or lysosomal acidification in the subject. In some embodiments, administration of the composition reduces and/or prevents biofilm formation in the subject.

Other aspects and embodiments of the disclosure will be apparent in light of the following detailed description.

### BRIEF DESCRIPTION OF THE DRAWINGS

FIGS. 1A-1C: DosRS is required for the adaptation and drug tolerance of M. abscessus under hypoxia. (A) Quantitative reverse-transcription PCR showing a strong upregulation of the dosR gene in Mabs ATCC 19977 grown for 24 h under microaerophilic conditions in standing T25 vented tissue culture flasks compared to well-aerated shaking flasks. DosR cDNA was normalized internally to the sigA cDNA in the same sample and the dosR mRNA level under microaerophilic conditions is expressed relative to the mRNA level of this gene under oxygenated conditions arbitrarily set to 1. Ratios of dosR/sigA mRNA are means±SD (n=3 RNA extractions and RT-qPCR reactions). (B) Comparative survival curves of the WT, dosRS mutant and the dosR and dosRS complemented mutant strains in the Wayne model. Decolorization of methylene blue occurred on d 5 indicative of oxygen depletion. The results presented are the means (±SD) of triplicate cultures and are representative of two independent experiments. Asterisks denote statistically significant differences between culture conditions pursuant to the Student's t-test (*p<0.05; ***p<0.0005). (C) Following 5 d of culture in the Wayne model, treatment with AMK (64 mg L⁻¹), AZI (32 mg L⁻¹) and CFZ (2 mg L⁻¹) for 7 d reduces the viability of the dosRS knock-out mutant but not that of the WT and dosRS complemented mutant strains. Treatment with IMI (32 mg L⁻¹) did not reduce the viability of WT or mutant strains. The results presented are the means (±SD) of triplicate cultures from one experiment and are representative of two independent experiments. Asterisks denote statistically significant differences between culture conditions pursuant to the Student's t-test (*p<0.05; **p<0.005).

FIGS. 2A-2E: Impact of DosRS deficiency on M. abscessus biofilm formation. Biofilm formation after 5 d of growth in SCFM medium in poly-D-lysine-coated microplates was determined by crystal violet staining (A) and CFU counting (B). The means and standard deviations of triplicate wells are shown. Asterisks denote statistically significant differences between WT and recombinant strains as determined by the Student's t-test (**p<0.005). (C) 2.5D rendering of the biofilms formed by WT Mabs ATCC 19977, MabsΔdosRS and the dosRS complementant expressing mCherry as imaged with fluorescence confocal microscopy. (D) Side view (XZ) of the biofilms formed by WT Mabs ATCC 19977, MabsΔdosRS and the dosRS complementant expressing mCherry as imaged using a KEYENCE BZ-X700 fluorescence microscope. (E) Biofilm thickness for each strain as measured after 5 d of growth in SCFM by 3D confocal imaging. Shown are the means and standard deviations of triplicate wells. Asterisks denote statistically significant differences between WT and recombinant strains pursuant to the Student's t-test (**p<0.005). All results are representative of two to three independent experiments.

FIG. 3: Infection of SCID mice with Mabs ATCC 19977 WT. the dosRS KO mutant and the dosRS complemented mutant. SCID mice were infected intravenously with 1.0×10⁶ CFU per animal of either Mabs ATCC 19977 WT, the dosRS KO mutant or the dosRS complemented mutant. AMK and AZI treatment of each group of mice began 28 d after infection, upon establishment of chronic infection. As of d 28, mice were treated daily (7 d/week) for 28 d with saline (gavage), AMK (150 mg/kg, subcutaneous injection) or AZI (200 mg/kg, gavage). Groups of mice were ethically euthanized on d 2, 28 and 56, and lungs and spleens were taken for bacterial enumeration (CFU). For each bacterial strain, the CFU results represent the average of 5 mice per time point, and bacterial loads are expressed as Log₁₀ CFU (±SEM). Asterisks denote statistically significant differences between antibiotic-treated WT and mutant- or complemented mutant-infected mice pursuant to the Student's t-test (p*<0.05; p**<0.005).

FIGS. 4A-4C: Effect of DosS inhibitors on MABSC survival and drug tolerance under hypoxia. (A) Structures of the DosS inhibitors used in this study. (B) Comparative survival of the WT, dosRS mutant and dosRS complemented mutant exposed to ART and OZ439 (40 μM each) in the Wayne model. Decolorization of methylene blue occurred on d 5 indicative of oxygen depletion. DosS inhibitor treatment was applied from the time of inoculation. CFU were plated for counting 7 d after the onset of hypoxia. The results presented are the means (±SD) of triplicate cultures and are representative of two independent experiments. Asterisks denote statistically significant differences between inhibitor-treated and non-treated cultures pursuant to the Student's t-test (**p<0.005; ***p<0.0005). (C) Mabs ATCC 19977 WT was cultured in the Wayne model in the presence of ART or OZ439 (40 μM each) from the time of inoculation. As cultures reached hypoxia. antibiotics, and more DosS inhibitors (40 μM each) were added and the cultures were incubated for another 7 d prior to CFU plating. The results presented are the means (±SD) of triplicate cultures and are representative of two independent experiments. Asterisks denote statistically significant differences between inhibitor-treated and non-treated cultures pursuant to the Student's t-test (*p<0.05; **p<0.005).

FIGS. 5A-5C: Effect of DosS inhibitors on MABSC biofilm formation. (A) Biofilm formation of ART-, AS-, OZ439- and OZ277-treated WT Mabs ATCC 19977 cultures after 5 d of growth in SCFM medium in poly-D-lysine-coated microplates as determined by crystal violet staining. The DosS inhibitors were added to the culture medium at the indicated concentrations on the first day and maintained throughout the duration of the experiment. The results presented are the means (±SD) of quadruplicate wells from one experiment and are representative of three independent experiments. Asterisks denote statistically significant differences between inhibitor-treated and non-treated cultures pursuant to the Student's t-test (*p<0.05; **p<0.005; ***p<0.0005; ****p<0.00005). (B) 2.5D rendering of biofilms formed by WT Mabs ATCC 19977 cultures either untreated or treated with ART (100 μM), OZ277 (25 or 100 μM) or OZ439 (25 or 100 μM) after 5 d of growth in SCFM. (C) Biofilm thickness of untreated and inhibitor-treated WT Mabs ATCC 19977 as measured after d 5 of growth in SCFM by 3D confocal imaging. Shown are the means and standard deviations of triplicate wells. Asterisks denote statistically significant differences between untreated and inhibitor-treated cultures pursuant to the Student's t-test (*p<0.05, **p<0.005, ***p<0.001). ART, artemisinin; AS, artesunate.

FIGS. 6A-6B: Effect of disrupting dosRS and of DosS inhibitor treatment on the transcriptional profile of Mabs ATCC 19977. (A) Differential gene expression between Mabs ATCC 19977 and MabsΔdosRS under microaerophilic conditions. Values are expressed as log₂ FC vs counts per million (CPM). Genes in red have a p-value <0.05 and the differential expression of five of them was further confirmed by RT-qPCR (see FIG. 17). (B) Venn-diagram showing genes downregulated in Mabs ATCC 19977 WT by ART or OZ439 treatment (log₂FC<−1, p-value<0.05) compared to genes downregulated under microaerophilic conditions in MabsΔdosRS (log₂FC<−2, p-value<0.05). (The complete list of these genes can be made available upon request.)

FIG. 7: Inhibition of the sensor kinase heme of DosS by ART, OZ439 and MSU-39446. UV-visible spectra of DosS showing treatment with dithionite (DTN) reduces the heme, which is the “on” state for the kinase. ART, OZ439 and MSU-39446, but not the degassed DMSO control, oxidize the heme.

FIGS. 8A-8C: Therapeutic efficacy and adjunct therapeutic efficacy of OZ439 in acutely and chronically MABSC-infected SCID mice. (A) Exposure of OZ439 in Mabs ATCC 19977-infected SCID mice. In the acute model, mice received OZ439 orally (200 mg/kg) every other d for 14 d. In the chronic model, mice received OZ439 orally (50 mg/kg) every other d for 28 d. Shown are the averages (±SD) of lung and plasma concentrations 2 and 48 hs after the last dosing. Two mice were used per time point in the acute model. Four mice were used per time point in the chronic model. (B) Therapeutic efficacy testing in an acute SCID mouse model of MABSC infection. Mice (n=5 mice/group) were infected intratracheally with 1.0×10⁶ CFUs of WT Mabs ATCC 19977. On d 12 post-infection, during the acute phase of infection, mice were treated daily (7 d/week) for 14 d, or every other day for OZ439, with vehicle used in the formulation of OZ439 (HPMC-SV), OZ439, AMK, AZI, IMI, CFZ, and the same four antibiotic treatments in combination with OZ439. See text for more details about the treatments. Bacterial loads were determined in the lungs, liver and spleen on d 2, 12 and 26. (C) Therapeutic efficacy testing in a chronic SCID mouse model of MABSC infection. Mice (five animals per group) were infected intravenously with 1.0×10⁶ CFUs of WT Mabs ATCC 19977. On d 28 post-infection, upon establishment of chronic infection, mice were treated daily (7 d/week) for 28 d, or every other day for OZ439, with formulation vehicle, OZ439, AMK, AZI, IMI and CFZ, and the same four antibiotics in combination with OZ439. See text for more details about the treatments. Bacterial loads were determined in the lungs, liver, and spleen on d 2, 28 and 56. In (B) and (C), asterisks denote statistically significant differences between antibiotic-treated and antibiotic+OZ439-treated mice, or untreated and OZ439-treated mice pursuant to the Student's t-test (*p<0.05; **p<0.005; ***p<0.0005).

FIG. 9: Kinetics of expression of the dosR and dosS genes under microaerophilic conditions in WT Mabs ATCC 19977 and the ΔdosRS knock-out mutant complemented with dosRS. Quantitative reverse-transcription PCR showing a strong upregulation of the dosR and dosS genes in Mabs ATCC 19977 (WT strain: left panel; ΔdosRS mutant complemented with dosRS: right panel) grown under microaerophilic conditions in standing T25 vented tissue culture flasks compared to well-aerated shaking flasks, with the expression of both genes peaking in the WT strain after 24 hours. DosR and DosS cDNA was normalized internally to the sigA cDNA in the same sample and the dosR and dosS mRNA levels under microaerophilic conditions are expressed relative to the mRNA level of these genes under oxygenated conditions after 3 hours of incubation arbitrarily set to 1. Ratios of dosR/sigA and dosS/sigA mRNA are means±SD (n=3 RNA extractions and RT-qPCR reactions).

FIG. 10: Gene replacement at the dosRS locus of Mabs ATCC 19977. Allelic replacement at the dosRS locus was confirmed by PCR using sets of primers (KOFw and KORv) located outside the linear allelic exchange substrate. The expected sizes of the products for the WT and mutant are indicated.

FIGS. 11A-11B: Loss of viability of MabsΔdosRS in the Wayne model. Flow cytometry analysis of mCherry-expressing WT Mabs ATCC 19977, MabsΔdosRS and MabsΔdosRS complemented with dosRS bacilli grown under hypoxic conditions in the Wayne model. (A) Representative histograms showing the proportion of mCherry-expressing (mCherry pos) and non-expressing (mCherry neg) bacilli for each strain on the day the cultures reached hypoxia (day 0; left panel) and after 21 days of incubation under hypoxic conditions (day 21, right panel). (B) Quantification of mCherry-positive bacilli on day 0 and day 21. The results show a clear decrease in the viability of MabsΔdosRS relative to the other strains after 21 days of hypoxic growth. The results presented are the means (±SD) of triplicate cultures. Asterisks denote statistically significant differences pursuant to the unpaired Student's t-test (**p<0.005).

FIG. 12: Effect of knocking-out dosRS on M. abscessus lipid biosynthesis. Mabs ATCC 19977 WT and dosRS KO were metabolically labeled with [1,2-¹⁴C]acetic acid for 24 hours in Dubos-Tween albumin broth under microaerophilic conditions in standing T25 vented flasks. Extracted total lipids from each strain (1,500 dpm per lane) were analyzed by TLC using different solvent systems to resolve lipids of different polarities (from left to right: [hexane:diethyl ether:acetic acid; 70:30:1 by vol.]; [chloroform:methanol:water; 20:4:0.5 by vol.]; [chloroform:methanol:water: 60:30:6 by vol.]). TAG (triacylglycerides); DAG, diacylglycerides; TDM, trehalose dimycolates; TMM, trehalose monomycolates; PE, phosphatidylethanolamine; CL, cardiolipin; PIM2, phosphatidyl-myo-inositol dimannosides. The results shown are representative of two independent experiments.

FIG. 13: Growth characteristics of Mabs ATCC 19977 WT, the dosRS KO and the dosR and dosRS complemented mutant strains in SCFM at 37° C. The results shown are representative of two independent experiments.

FIG. 14: Effect of DosS inhibitors on the drug tolerance of MabsΔdosRS under hypoxia. The Mabs dosRS mutant was cultured in the Wayne model in the presence of DMSO, ART or OZ439 (40 μM each) from the time of inoculation. As cultures reached hypoxia, antibiotics [AMI (64 mg L⁻¹) and AZI (32 mg L⁻¹)] and more DMSO or DosS inhibitors (40 μM each) were added and the cultures were incubated for another 7 days prior to CFU plating. As expected, DosS inhibitors had no significant effect (per the unpaired Student's t-test; p<0.05) on the susceptibility of the dosRS mutant to AMI or AZI indicative of the on-target activity of ART and OZ439. The results presented are the means (±SD) of triplicate cultures.

FIG. 15: Effect of DosS inhibitors on MABSC biofilm dispersal. Four-day-old biofilms were washed with PBS and DosS inhibitors (or 0.05% DMSO as control) in PBS were added at the indicated concentrations (in μM). The plate was incubated for another 24 h at 37° C. at which point biofilms were quantified by crystal violet staining and the turbidity of planktonic bacteria released in the medium assessed spectrophotometrically at 600 nm. The results presented are the means (±SD) of quadruplicate wells and are representative of two independent experiments.

FIG. 16: Identification of the Mabs DosR DNA-binding site. Logo of the DosR-binding motif in Mabs ATCC 19977, based on 13 sites found in the promoter regions of DosR-regulated genes, including MAB_0156c, MAB_1040, MAB_1041, MAB_1042c, MAB_2489, MAB_3134c, MAB_3354, MAB_3891c, MAB_3902c and MAB_3903. The sequence was constructed using the WebLogo tool.

FIG. 17: Transcriptional profiles of ART- and OZ439-treated WT Mabs ATCC 19977 cells and of the dosRS KO mutant under microaerophilic growth conditions. RNA-seq (log₂ fold-change) (red) and quantitative reverse transcription-PCR (RT-qPCR) (grey) show five genes expressed at lower level in the dosRS KO mutant or in ART- and OZ439-treated Mabs ATCC 19977 cells compared to untreated WT cells: MAB 1042c (ctaD, cytochrome c oxidase polypeptide I), MAB 2489 (usp, universal stress protein), MAB 3354 (desA1, desaturase). MAB_3902c (putative aminoglycoside phosphotransferase) and MAB_3903 (putative Acg nitroreductase). All bacterial cultures were grown for 24 hours under microaerophilic conditions in standing T25 vented tissue culture flasks. Ratios of genes/sigA mRNA are means f standard deviations (n=3 RNA extractions and RT-qPCR reactions).

FIG. 18: Effect of DosS inhibitors on NO signaling. Mabs ATCC 19977 was grown to OD ˜0.3 in 7H9-ADC-Tween 80, treated for 1 h with either DMSO (control) or 50 μM of DosS inhibitors, followed by addition of Spermine NONOate (50 μM final concentration). Cells were further incubated with shaking for 40 min, collected and processed for quantitative reverse-transcription PCR of the dosR gene. dosR cDNA was normalized internally to the sigA cDNA in the same sample and dosR mRNA levels are expressed relative to the mRNA level of this gene in the absence of Spermine NONOate in the medium (arbitrarily set to 1). Ratios of dosR/sigA mRNA are means±SD (n=3 RNA extractions and RT-qPCR reactions).

FIGS. 19A-19B: Therapeutic efficacy of OZ277 in acutely MABSC-infected SCID mice. (A) Exposure of OZ277 in Mabs ATCC 19977-infected SCID mice. Mice received OZ277 orally (200 mg/kg) daily for 14 days. Shown are the averages (±SD) of lung and plasma concentrations 2 and 24 hours after the last dosing. Two mice were used per time point. (B) Therapeutic efficacy testing in an acute SCID mouse model of MABSC infection. Mice infected intratrachcally with 10 CFUs of WT Mabs ATCC 19977 were treated daily as of day 12 for 14 days with HPMC-SV vehicle, OZ277 (200 mg/kg, gavage), AMK (150 mg/kg, subcutaneous injection), AZI (200 mg/kg, gavage), IMI (100 mg/kg, subcutaneous injection), CFZ (20 mg/kg, gavage), and the same four antibiotic treatments in combination with OZ277. Bacterial loads were determined in the lungs, liver and spleen on days 2, 12 and 26. Asterisks denote statistically significant differences between untreated and OZ277-treated mice (*p<0.05; ***p<0.0005). No statistically significant differences were noted between antibiotic-treated and antibiotic+OZ277-treated mice pursuant to the Student's t-test (p<0.05).

FIGS. 20A-20B: Therapeutic efficacy and adjunct therapeutic efficacy of OZ439 (A) and OZ277 (B) in acutely MABSC-infected SCID mice. (A) OZ439 therapeutic efficacy testing in an acute SCID mouse model of MABSC infection. Mice (n=5 mice/group) were infected intratracheally with 1.0×10⁶ CFUs of WT Mabs ATCC 19977. On day 12 post-infection, during the acute phase of infection, mice were treated daily (7 days/week) for 14 days, or every other day for OZ439, with vehicle used in the formulation of OZ439 (25% propylene glycol in water), OZ439 (20 mg/kg; delivered through the intratracheal route), AMK. AZI, IMI, CFZ, (AMK+AZI+IMI+CFZ=“combo”) and the same five antibiotic treatments in combination with OZ439. See text for more details about the antibiotic treatments. Bacterial loads were determined in the lungs, liver and spleen on days 2, 12 and 26. (B) OZ277 therapeutic efficacy testing in an acute SCID mouse model of MABSC infection. Mice (n=5 mice/group) were infected intratracheally with 1.0×10⁶ CFUs of WT Mabs ATCC 19977. On day 12 post-infection, during the acute phase of infection, mice were treated daily (7 days/week) for 14 days with vehicle used in the formulation of OZ277 (25% propylene glycol in water), OZ277 (20 mg/kg; delivered through the intratracheal route), AMK, AZI, IMI, CFZ, (AMK+AZI+IMI+CFZ=“combo”) and the same five antibiotic treatments in combination with OZ277. See text for more details about the antibiotic treatments. Bacterial loads were determined in the lungs, liver and spleen on days 2, 12 and 26. In (A) and (B), asterisks denote statistically significant differences between antibiotic-treated and antibiotic+OZ277/OZ439-treated mice, or untreated and OZ277/OZ439-treated mice pursuant to the Student's t-test (*p<0.05; **p<0.005; ***p<0.0005).

FIG. 21: THP-1 cells were plated at 1×10⁶ cells per ml in 24-well plates and treated for 24 hrs. with 10 ng/ml PMA for differentiating to macrophages. PMA medium was removed, and cells were cultured for an additional 24 hrs. Cells were then treated with either nothing, 300 ng/ml LPS or either Mabs ATCC19977 (smooth) or Mabs ATCC19977 (rough)(aka CipR) at an MOI of 5. After 9 h, the medium was removed and replaced with medium containing either nothing or 6 μM of OZ439 in triplicate wells and THP-1 cells were cultured for an additional 30 hr. Supernatants were harvested and assayed for NO production by the Griess Assay. Half the cells were stained with H2DCFDA for ROS analysis and half were stained for macrophage activation markers and analyzed by flow cytometry. Statistical differences between treatment groups were determined by one way ANOVA with Dunnett's multiple comparison test (*=p<0.05; ***=p<0.001; ****=p<0.0001).

### DETAILED DESCRIPTION

A search for alternative Mycobacterium abscessus treatments led to an investigation of the two-component regulator DosRS which, in Mycobacterium tuberculosis, is required for the bacterium to establish a state of non-replicating, drug-tolerant persistence in response to a variety of host stresses. Experiments of the present disclosure demonstrate that the genetic disruption of dosRS impairs the adaptation of M. abscessus to hypoxia, resulting in decreased bacterial survival following oxygen depletion, reduced tolerance to a number of antibiotics in vitro and in vivo, and the inhibition of biofilm formation. It was determined that three antimalarial drugs or drug candidates, artemisinin, OZ277, and OZ439, target DosS-mediated hypoxic signaling in M. abscessus and recapitulate the phenotypic effects of genetically disrupting dosS. Importantly, OZ439 displayed bactericidal activity comparable to standard-of-care antibiotics in chronically infected mice, in addition to potentiating the activity of antibiotics used in combination. The identification of antimalarial drugs as potent inhibitors and adjunct inhibitors of M. abscessus in vivo offers repurposing opportunities that could have an immediate impact in the clinic.

As described further herein, the existence of different microenvironments within a host is likely to drive intra- and extracellular NTM into distinct physiological states and may explain why NTM infections are usually incurable with antibiotic therapy alone and why adjunctive surgical resection of infected tissues may improve cure. MABSC bacilli located inside activated macrophages, in avascular necrotic granulomas, and within biofilms are exposed to O₂ depletion and NO-induced hypoxia. Results of the present disclosure demonstrate that a critical element of the adaptation of MABSC to these stresses is the two-component regulatory system DosRS. Unlike the situation in Mtb in which DosS is not critical to the activation of the DosR regulon, most likely as a result of the existence of a compensatory sensor kinase (DosT), a functional DosS is essential to the induction of DosR upon O₂ depletion in MABSC. In line with this observation, inhibitors targeting the sensor heme of MABSC DosS phenocopy the effects of genetically disrupting dosRS or dosS in terms of inhibition of biofilm formation and decreased survival and drug tolerance under hypoxia.

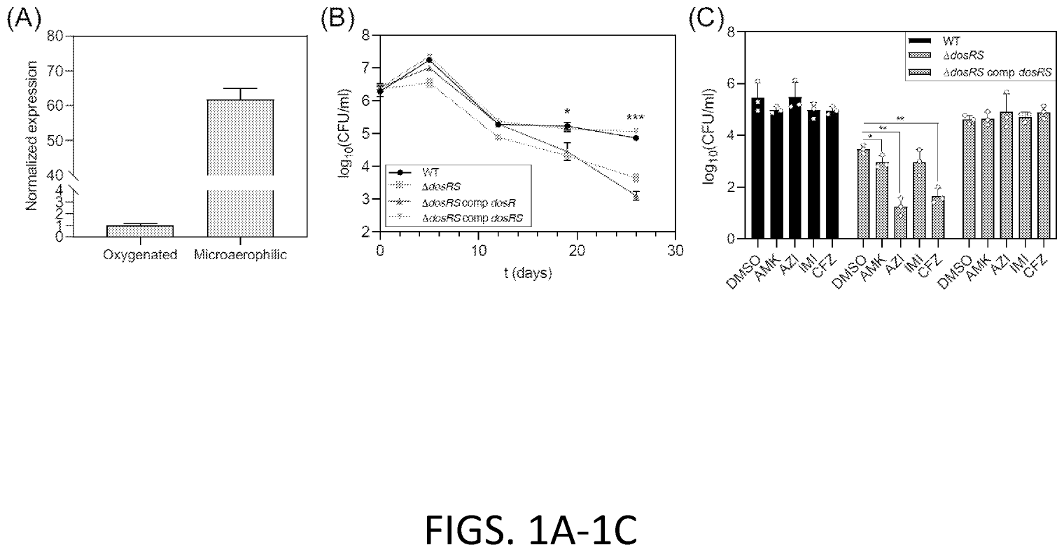

RNA-seq analysis of the MABSC DosR regulon provided insights into the metabolic adaptation driving the ability of the bacilli to maintain energy levels and a redox balance in the absence of respiration. Other differentially expressed genes of interest between the WT and dosRS KO mutant include genes expected to promote intracellular survival by protecting MABSC from host-reactive nitrogen intermediates and reactive oxygen species (e.g., MAB_0894c, MAB_3134c, MAB_2530c), a DosRS regulon gene controlling polyphosphate concentrations in the cells (e.g., MAB_1040). which is likely to play a role in biofilm development, and two Mtb-conserved genes proposed to be involved in aminoglycoside resistance (e.g., MAB_3902c and MAB_2489) that may account for the potentiation of the activity of AMK against the dosRS mutant and DosS inhibitor-treated WT bacilli in vitro and in vivo (see FIGS. 1, 4 and 8).

In addition to the above, results of the present disclosure indicated an unexpected and significant reduction in MABSC loads following treatment with OZ439 or OZ277 individually, in both acutely and chronically infected mice. For example, as described further herein, OZ439 (e.g., when given orally at 200 mg/kg) displayed an efficacy comparable to that of CFZ and IMI, and its efficacy was comparable to that of all four antibiotics tested herein (AMK, AZI, CFZ and IMI) in the chronic model despite reduced dosing (e.g., 50 mg/kg). These results demonstrate that OZ439 is most active during the chronic phase of infection.

Multiple factors may explain the dramatic decrease in survival of DosS inhibitor-treated WT Mabs in vivo whereas the dosRS KO mutant failed to show any virulence attenuation in the same mouse model. One important difference between the two studies resides in the differential effects of infecting mice with an MABSC strain devoid of DosR and DosS proteins from the onset of infection, and an MABSC strain that produces both proteins but whose DosS activity is pharmacologically and partially inactivated once the infection is established. In Mtb, the production of DosR-regulated antigens (which would be expressed in a WT strain before the initiation of OZ439 treatment but not in a dosRS deletion mutant) has been implicated in the magnitude and timing of adaptive immune responses. DosS from Mtb has further been associated with DosR-independent activities such as the phosphorylation of a subset of proteins that may also impact the course of infection. Dissimilarities in antigen repertoires and bacterial adaptations to the host could account for differences in the susceptibility of the dosRS KO and DosS inhibitor-treated WT bacilli to immune clearance, including in SCID mice that lack the ability to mount normal adaptive immune responses. In spite of the relative specificity of ART and OZ439 for DosS at the concentrations used in the RNA-seq analysis, it is possible that these compounds further display off-target bactericidal activity in vivo, especially at the relatively high concentrations achieved by OZ439 in the lungs of infected SCID mice. Alternatively, the activity of OZ439 and OZ277 could be host-directed, which is further supported by the ability of artemisinins to enhance autophagy and lysosomal killing. Determining whether OZ277 and OZ439 share this activity with ART and to what extent this activity might contribute to the intracellular killing of MABSC would be of great interest in further deciphering the therapeutic properties of these compounds.

Taken together, results of the present disclosure have demonstrated that orally available antimalarial drugs and drug candidates have the potential to improve MABSC treatment both as a stand-alone therapy and when used by in combination with standard-of-care antibiotics. The repurposing of these agents provides a much-needed opportunity for innovative combination chemotherapy that could be available in the foreseeable future for patients suffering from MABSC infections.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions. will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the present disclosure may be readily combined, without departing from the scope or spirit of the embodiments provided herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., non-tuberculous mycobacterial infection). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition in the patient (e.g., non-tuberculous mycobacterial infection). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers. diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol. and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W. Remington's Pharmaceutical Sciences, Easton Pa., Mack Publishing Company, 19.sup.th ed., 1995) describes formulations that can be used in connection with the subject invention.

Formulations suitable for nebulizing administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

An “effective amount” of an agent, e.g., a pharmaceutical formulation, as used herein generally refers to an amount effective, at dosages and for periods of time necessary, to achieve the desired therapeutic or prophylactic result.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions

Embodiments of the present disclosure provide compositions and methods related to the treatment of non-tuberculous mycobacterial infections in a subject. In particular, the present disclosure provides compositions comprising an anti-malarial agent and associated methods of treating and/or preventing a non-tuberculous mycobacterial infection in a subject using the composition. As described further herein, embodiments of the present disclosure include a composition for treating and/or preventing a non-tuberculous mycobacterial infection in a subject comprising a therapeutically effective dose of at least one anti-malarial agent and a pharmaceutically acceptable carrier.

In some embodiments, the non-tuberculous mycobacterial infection is caused by a non-tuberculous mycobacteria (NTM) selected from the group consisting of: M. abscessus subsp. abscessus, M. abscessus subsp. massiliense, M. abscessus subsp. bolletii, Mycobacterium avium complex, M. kansasii, M. chelonae, M. fortuitum, and M. xenopi, M. peregrinum, M. mucogenicum, M. senegalense, M. immunogenum, M. simiae, M. gordonae, M. septicum, M. malmoense, M. goodii, M. haemophilum, M. genavense, M. scrofulaceum, M. lentiflavum, M. bohemicum, and M. marinum, including any combination thereof. In some embodiments, the non-tuberculous mycobacterial infection is caused by a mycobacterium selected from the group consisting of: M. leprae, and M. ulcerans, including a combination thereof.

In some embodiments, the composition treats and/or prevents at least one symptom of the non-tuberculous mycobacterial infection in the subject. As would be recognized by one of ordinary skill in the art based on the present disclosure, symptoms experienced by a subject with a non-tuberculous mycobacterial infection can vary, depending on a variety of factors. Symptoms can include, but are not limited to, a cough, weight loss, appetite loss, fatigue, shortness of breath, a fever, and night sweats. In some embodiments, the one or more symptoms of the non-tuberculous mycobacterial infection are reduced, inhibited, and/or ameliorated by the compositions of the present disclosure.

In some embodiments, the compositions of the present disclosure include at least one anti-malarial agent. In some embodiments, the at least one anti-malarial agent comprises a trioxolane (secondary ozonide). In some embodiments, the at least one anti-malarial agent comprises a synthetic peroxide. In some embodiments, the at least one anti-malarial agent comprises arterolane (OZ277). In some embodiments, the at least one anti-malarial agent consists essentially of arterolane (OZ277). In some embodiments, the at least one anti-malarial agent comprises ozonide OZ439. In some embodiments, the at least one anti-malarial agent consists essentially of ozonide OZ439. In some embodiments, the at least one anti-malarial agent comprises OZ277 and OZ439. In some embodiments, the at least one anti-malarial agent consists essentially of OZ277 and OZ439. As would be recognized by one of ordinary skill in the art based on the present disclosure, the compositions described herein can include OZ277 and/or OZ439, as well as other components (e.g., adjuvants, excipients, buffers, carrier, etc.) that together can form a pharmaceutical composition for the treatment and/or prevention of a non-tuberculous mycobacterial infection (or a symptom caused by a non-tuberculous mycobacterial infection).

In accordance with the above embodiments, the compositions of the present disclosure include at least one anti-malarial agent that is present in the composition in a therapeutically effective amount or dose. In some embodiments, the at least one anti-malarial agent (e.g., OZ277 and/or OZ439) is present in the composition in an amount ranging from about 5 mg to about 1200 mg. In some embodiments, the at least one anti-malarial agent (e.g., OZ277 and/or OZ439) is present in the composition in an amount ranging from about 50 mg to about 1200 mg. In some embodiments, the at least one anti-malarial agent (e.g., OZ277 and/or OZ439) is present in the composition in an amount ranging from about 100 mg to about 1200 mg. In some embodiments, the at least one anti-malarial agent (e.g., OZ277 and/or OZ439) is present in the composition in an amount ranging from about 250 mg to about 1200 mg. In some embodiments, the at least one anti-malarial agent (e.g., OZ277 and/or OZ439) is present in the composition in an amount ranging from about 500 mg to about 1200 mg. In some embodiments, the at least one anti-malarial agent (e.g., OZ277 and/or OZ439) is present in the composition in an amount ranging from about 750 mg to about 1200 mg. In some embodiments, the at least one anti-malarial agent (e.g., OZ277 and/or OZ439) is present in the composition in an amount ranging from about 1000 mg to about 1200 mg. In some embodiments, the at least one anti-malarial agent (e.g., OZ277 and/or OZ439) is present in the composition in an amount ranging from about 5 mg to about 1000 mg. In some embodiments, the at least one anti-malarial agent (e.g., OZ277 and/or OZ439) is present in the composition in an amount ranging from about 5 mg to about 750 mg. In some embodiments, the at least one anti-malarial agent (e.g., OZ277 and/or OZ439) is present in the composition in an amount ranging from about 5 mg to about 500 mg. In some embodiments, the at least one anti-malarial agent (e.g., OZ277 and/or OZ439) is present in the composition in an amount ranging from about 5 mg to about 250 mg. In some embodiments, the at least one anti-malarial agent (e.g., OZ277 and/or OZ439) is present in the composition in an amount ranging from about 5 mg to about 100 mg. In some embodiments, the at least one anti-malarial agent (e.g., OZ277 and/or OZ439) is present in the composition in an amount ranging from about 100 mg to about 1000 mg. In some embodiments, the at least one anti-malarial agent (e.g., OZ277 and/or OZ439) is present in the composition in an amount ranging from about 250 mg to about 750 mg. In some embodiments, the at least one anti-malarial agent (e.g., OZ277 and/or OZ439) is present in the composition in an amount ranging from about 250 mg to about 500 mg. In some embodiments, the at least one anti-malarial agent (e.g., OZ277 and/or OZ439) is present in the composition in an amount ranging from about 500 mg to about 750 mg.

In some embodiments, the compositions of the present disclosure include OZ277 as the at least one anti-malarial agent that is present in the composition in a therapeutically effective amount or dose. In some embodiments, OZ277 is present in the composition in an amount ranging from about 5 mg to about 1200 mg. In some embodiments, OZ277 is present in the composition in an amount ranging from about 50 mg to about 1200 mg. In some embodiments, OZ277 is present in the composition in an amount ranging from about 100 mg to about 1200 mg. In some embodiments, OZ277 is present in the composition in an amount ranging from about 250 mg to about 1200 mg. In some embodiments, OZ277 is present in the composition in an amount ranging from about 500 mg to about 1200 mg. In some embodiments, OZ277 is present in the composition in an amount ranging from about 750 mg to about 1200 mg. In some embodiments, OZ277 is present in the composition in an amount ranging from about 1000 mg to about 1200 mg. In some embodiments, OZ277 is present in the composition in an amount ranging from about 5 mg to about 1000 mg. In some embodiments, OZ277 is present in the composition in an amount ranging from about 5 mg to about 750 mg. In some embodiments, OZ277 is present in the composition in an amount ranging from about 5 mg to about 500 mg. In some embodiments, OZ277 is present in the composition in an amount ranging from about 5 mg to about 250 mg. In some embodiments, OZ277 is present in the composition in an amount ranging from about 5 mg to about 100 mg. In some embodiments, OZ277 is present in the composition in an amount ranging from about 100 mg to about 1000 mg. In some embodiments, OZ277 is present in the composition in an amount ranging from about 250 mg to about 750 mg. In some embodiments, OZ277 is present in the composition in an amount ranging from about 250 mg to about 500 mg. In some embodiments, OZ277 is present in the composition in an amount ranging from about 500 mg to about 750 mg.

In some embodiments, the compositions of the present disclosure include OZ439 as the at least one anti-malarial agent that is present in the composition in a therapeutically effective amount or dose. In some embodiments, OZ439 is present in the composition in an amount ranging from about 5 mg to about 1200 mg. In some embodiments, OZ439 is present in the composition in an amount ranging from about 50 mg to about 1200 mg. In some embodiments, OZ439 is present in the composition in an amount ranging from about 100 mg to about 1200 mg. In some embodiments, OZ439 is present in the composition in an amount ranging from about 250 mg to about 1200 mg. In some embodiments, OZ439 is present in the composition in an amount ranging from about 500 mg to about 1200 mg. In some embodiments, OZ439 is present in the composition in an amount ranging from about 750 mg to about 1200 mg. In some embodiments, OZ439 is present in the composition in an amount ranging from about 1000 mg to about 1200 mg. In some embodiments, OZ439 is present in the composition in an amount ranging from about 5 mg to about 1000 mg. In some embodiments. OZ439 is present in the composition in an amount ranging from about 5 mg to about 750 mg. In some embodiments, OZ439 is present in the composition in an amount ranging from about 5 mg to about 500 mg. In some embodiments, OZ439 is present in the composition in an amount ranging from about 5 mg to about 250 mg. In some embodiments, OZ439 is present in the composition in an amount ranging from about 5 mg to about 100 mg. In some embodiments, OZ439 is present in the composition in an amount ranging from about 100 mg to about 1000 mg. In some embodiments, OZ439 is present in the composition in an amount ranging from about 250 mg to about 750 mg. In some embodiments, OZ439 is present in the composition in an amount ranging from about 250 mg to about 500 mg. In some embodiments, OZ439 is present in the composition in an amount ranging from about 500 mg to about 750 mg.

In some embodiments, the compositions of the present disclosure include at least one antibiotic in addition to the at least one anti-malarial agent. In some embodiments, the at least one antibiotic includes, but is not limited to, clarithromycin, azithromycin, rifampin, rifabutin, ethambutol, streptomycin, amikacin, ciprofloxacin, doxycycline, clofazimine, isoniazid, linezolid, moxifloxacin, trimethoprim/sulfamethoxazole, cefoxitin, imipenem, and tigecycline, and any combination thereof. The antibiotic can be present in the composition at a therapeutically effective amount or dose (e.g., 10 mg to 1000 mg), as would be recognized by one of ordinary skill in the art based on the present disclosure. In some embodiments, the at least one antibiotic is clarithromycin. In some embodiments, the at least one antibiotic is azithromycin. In some embodiments, the at least one antibiotic is rifampin. In some embodiments, the at least one antibiotic is rifabutin. In some embodiments, the at least one antibiotic is ethambutol. In some embodiments, the at least one antibiotic is streptomycin. In some embodiments, the at least one antibiotic is amikacin. In some embodiments, the at least one antibiotic is ciprofloxacin. In some embodiments. the at least one antibiotic is doxycycline. In some embodiments, the at least one antibiotic is clofazimine. In some embodiments, the at least one antibiotic is isoniazid. In some embodiments, the at least one antibiotic is linezolid. In some embodiments, the at least one antibiotic is moxifloxacin. In some embodiments, the at least one antibiotic is trimethoprim/sulfamethoxazole. In some embodiments, the at least one antibiotic is cefoxitin. In some embodiments, the at least one antibiotic is imipenem. In some embodiments, the at least one antibiotic is tigecycline.

In some embodiments. the antibiotic(s) is/are present in the composition at a lower dose than is typically considered a therapeutically effective dose because the anti-malarial agent present in the composition acts synergistically with the antibiotic(s) to treat and/or prevent a non-tuberculous mycobacterial infection. In some embodiments, and as described further herein, the compositions of the present disclosure do not include any antibiotic because the anti-malarial agent present in the composition treats and/or prevents a non-tuberculous mycobacterial infection without the need for an antibiotic.

The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein.

A subject may be treated with a composition of the present disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously. A beneficial pharmacokinetic profile can be obtained by the administration of a formulation or dosage form suitable for once, twice, or three times a day administration in an amount sufficient to provide a required dose of the composition. In some embodiments, the composition is administered in a single dose. In some embodiments, the composition is administered in multiple doses. In some embodiments, the composition is administered daily, weekly, bi-weekly or monthly.

Certain dosage forms and formulations may minimize the variation between peak and trough plasma and/or brain levels of compositions of the disclosure and in particular provide a sustained therapeutically effective amount of the compositions. The present disclosure also contemplates a formulation or dosage form comprising amounts of one or more composition of the disclosure that results in therapeutically effective amounts of the composition over a dosing period, in particular a 24 h dosing period. A medicament or treatment of the disclosure may comprise a unit dosage of at least one composition of the disclosure to provide therapeutic effects. A “unit dosage or “dosage unit” refers to a unitary (e.g., a single dose), which is capable of being administered to a subject, and which may be readily handled and packed, remaining as a physically and chemically stable unit dose comprising either the active agents as such or a mixture with one or more solid or liquid pharmaceutical excipients, carriers, or vehicles.

### 3. Methods of Administration

Embodiments of the present disclosure also include a method of treating and/or preventing a non-tuberculous mycobacterial infection in a subject. In accordance with these embodiments, the method includes administering any of the compositions described herein to the subject for the treatment and/or prevention of a non-tuberculous mycobacterial infection (or a symptom caused by a non-tuberculous mycobacterial infection). As would be recognized by one of ordinary skill in the art based on the present disclosure, the compositions provided herein can be formulated for administration by any medically appropriate means, including but not limited to, as a tablet, capsule, powder, granule, sachet, gel, liquid, spray, suspension, or inhalant. In some embodiments, the composition is administered to the subject orally, sublingually, topically, intravenously, subcutaneously, transcutaneously, nasally, vaginally, rectally, or by inhalation. In some embodiments, the composition is administered via nebulization to lung tissue.

In accordance with these embodiments, the compositions of the present disclosure can persist in the subject's tissues (e.g., lung tissue) for at least 72 hours after administration. In some embodiments, the plurality of EVs or exosomes persist in a subject for at least 12 hours, at least 24 hours, at least 36 hours, at least 48 hours, at least 60 hours, at least 72 hours, at least 84 hours, and at least 96 hours. In some embodiments, the compositions are administered every 24 hours, every 48 hours, every 72 hours, or every 96 hours, depending on the dose being administered and the subject's physiological characteristics. In some embodiments, the composition is administered in a single dose. In some embodiments, the composition is administered in multiple doses. In some embodiments, the composition is administered daily, weekly, bi-weekly or monthly.

In some embodiments, a single dose of the compositions of the present disclosure can exert a beneficial effect (e.g., treat and/or prevent a non-tuberculous mycobacterial infection or a symptom caused by a non-tuberculous mycobacterial infection) on a subject. In some embodiments, two or more doses are required to provide a beneficial effect. In some embodiments, three or more doses are required to provide a beneficial effect. In some embodiments, four or more doses are required to provide a beneficial effect. In some embodiments, five or more doses are required to provide a beneficial effect. In some embodiments, six or more doses are required to provide a beneficial effect. In some embodiments, seven or more doses are required to provide a beneficial effect. In some embodiments, eight or more doses are required to provide a beneficial effect. In some embodiments, nine or more doses are required to provide a beneficial effect. In some embodiments, ten or more doses are required to provide a beneficial effect.

In some embodiments, the present disclosure encompasses methods of treating a human subject that has a non-tuberculous mycobacterial infection or is susceptible of contacting a non-tuberculous mycobacterial infection. In some embodiments, the method comprises administering (e.g., orally or via inhalation) to the human subject a pharmaceutical composition comprising an anti-malarial agent (e.g., OZ277 and/or OZ439) in an amount effective in modulating a pathological condition when delivered to the human subject in need thereof. In some embodiments, the pathological condition is a non-tuberculous mycobacterial. The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein.

A subject may be treated with a composition of the present disclosure or composition or unit dosage thereof on substantially any desired schedule. They may be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously. A beneficial pharmacokinetic profile can be obtained by the administration of a formulation or dosage form suitable for once, twice, or three times a day administration in an amount sufficient to provide a required dose of the composition. Certain dosage forms and formulations may minimize the variation between peak and trough plasma and/or brain levels of compositions of the disclosure and in particular provide a sustained therapeutically effective amount of the compositions. The present disclosure also contemplates a formulation or dosage form comprising amounts of one or more compositions of the disclosure that results in therapeutically effective amounts of the composition over a dosing period (e.g., a 24 h dosing period). A medicament or treatment of the disclosure may comprise a unit dosage of at least one composition of the disclosure to provide therapeutic effects. A “unit dosage or “dosage unit” refers to a unitary (e.g., a single dose), which is capable of being administered to a subject, and which may be readily handled and packed, remaining as a physically and chemically stable unit dose comprising either the active agents as such or a mixture with one or more solid or liquid pharmaceutical excipients, carriers, or vehicles.

As described further herein, the compositions of the present disclosure can be administered to a subject at a dose ranging from about 50 mg/kg to about 1200 mg/kg. In some embodiments, the composition is administered at a dose ranging from about 100 mg/kg to about 1200 mg/kg. In some embodiments, the composition is administered at a dose ranging from about 250 mg/kg to about 1200 mg/kg. In some embodiments, the composition is administered at a dose ranging from about 500 mg/kg to about 1200 mg/kg. In some embodiments, the composition is administered at a dose ranging from about 750 mg/kg to about 1200 mg/kg. In some embodiments, the composition is administered at a dose ranging from about 50 mg/kg to about 1000 mg/kg. In some embodiments, the composition is administered at a dose ranging from about 50 mg/kg to about 750 mg/kg. In some embodiments, the composition is administered at a dose ranging from about 50 mg/kg to about 500 mg/kg. In some embodiments, the composition is administered at a dose ranging from about 50 mg/kg to about 250 mg/kg. In some embodiments, the composition is administered at a dose ranging from about 100 mg/kg to about 750 mg/kg. In some embodiments, the composition is administered at a dose ranging from about 250 mg/kg to about 500 mg/kg.

In some embodiments, the composition is administered to the subject in an amount ranging from about 50 mg/dose to about 1200 mg/dose. In some embodiments, the composition is administered to the subject in an amount ranging from about 100 mg/dose to about 1200 mg/dose. In some embodiments. the composition is administered to the subject in an amount ranging from about 250 mg/dose to about 1200 mg/dose. In some embodiments, the composition is administered to the subject in an amount ranging from about 500 mg/dose to about 1200 mg/dose. In some embodiments, the composition is administered to the subject in an amount ranging from about 750 mg/dose to about 1200 mg/dose. In some embodiments, the composition is administered to the subject in an amount ranging from about 50 mg/dose to about 1000 mg/dose. In some embodiments, the composition is administered to the subject in an amount ranging from about 50 mg/dose to about 750 mg/dose. In some embodiments, the composition is administered to the subject in an amount ranging from about 50 mg/dose to about 500 mg/dose. In some embodiments, the composition is administered to the subject in an amount ranging from about 50 mg/dose to about 250 mg/dose. In some embodiments, the composition is administered to the subject in an amount ranging from about 100 mg/dose to about 750 mg/dose. In some embodiments, the composition is administered to the subject in an amount ranging from about 250 mg/dose to about 500 mg/dose.

In some embodiments. the composition is administered with an antibiotic. In some embodiments, the composition is administered with the antibiotic in the same dose or in a different dose. In some embodiments, the composition is not administered with an antibiotic.

In accordance with these embodiments, administration of the composition enhances at least one of oxidative stress. apoptosis, autophagy and/or lysosomal acidification in the subject. In some embodiments, administration of the composition reduces and/or prevents biofilm formation in the subject.

### 4. Materials and Methods

Study Design. The objectives of this study were to assess the therapeutic potential of the DosRS two-component regulatory system of MABSC and to identify potent inhibitors of this regulator active in vivo. A dosRS KO mutant of Mabs ATCC 19977 was constructed. and this mutant was compared to its WT parental and complemented mutant strains for its ability to survive and develop drug tolerance under hypoxia, and form biofilms. The same KO mutant was compared to the WT parental strain for virulence and drug tolerance in a chronic SCID mouse model of MABSC infection. Subsequently, compounds known to inhibit DosS hypoxic signaling in Mtb and derivatives of these compounds with improved PK properties were tested for their ability to phenocopy the effect of genetically disrupting dosRS in vitro. Gene transcriptional profiling experiments and biochemical assays with compounds displaying activity in vitro confirmed that they abolish DosS signaling in MABSC. Finally, two of the compounds were evaluated for therapeutic and adjunct therapeutic efficacy in acute and/or chronic mouse models of MABSC infection. All protocols were approved by the Colorado State University institutional biosafety committee. All in vitro experiments were repeated at least two times. All in vivo procedures were reviewed and approved by the Colorado State University Animal Care and Usage Committee.

Strains and culture media. Mabs ATCC 19977 WT (smooth morphotype) and recombinant strains were grown under agitation at 37° C. in Middlebrook 7H9 medium supplemented with 10% albumin-dextrose-catalase (ADC) (BD Sciences) and 0.05% Tween 80, in cation-adjusted Mueller Hinton II broth (BD Sciences) with 0.05% tyloxapol, in SCFM, or on Middlebrook 7H11 agar supplemented with 10% oleic acid-albumin-dextrose-catalase (OADC) (BD Sciences). Zeocin (Zeo) and kanamycin (Kan) were added to the culture media at a final concentration of 100 mg L⁻¹ and 200 mg L⁻¹, respectively. For growth under microaerophilic and hypoxic conditions, Mabs was grown in Dubos-Tween albumin broth at a final pH of 7.25 either in standing T25 vented tissue culture flasks (microaerophilic conditions) or in 16×100 mm glass tubes with tightly sealed screw caps with rubber septa under constant stirring using Teflon-coated magnetic bars (hypoxic Wayne model). Decolorization of methylene blue (1.5 mg L⁻¹ final concentration) in control tubes served as a visual indication of oxygen depletion in the Wayne model.

MAbs dosRS knock-out mutant and complemented mutant strains. A variation of the katG-based counterselection method described by Sander and collaborator was used to generate a dosRS deletion mutant of Mabs ATCC 19977. The expression of the katG gene from Mtb in MABSC renders this species susceptible to isoniazid. Briefly, an allelic exchange substrate consisting of the zeocin-resistance cassette (zeo) and the colored marker xylE bracketed by ˜1,000 bp of upstream and downstream DNA immediately flanking the dosRS operon of Mabs ATCC 19977 (MAB_3891c-MAB_3890c) was cloned in pKATs, a derivative of the replicative E. coli-Myobacterium shuttle plasmid pMP349, harboring the Mtb katG gene. The resulting plasmid, pKATs-dosRS::zeo-xylE. was electrotransformed in Mabs ATCC 19977 and a transformant selected on zeocin-containing agar plates. Upon a culturing step in liquid broth, the transformant was finally plated on agar with zeocin and 512 mg L⁻¹ of isoniazid to select for allelic exchange mutants. Allelic replacement at the dosRS locus was verified by PCR and sequencing. For complementation, 220-bp of the promoter region of dosR and the entire coding sequence of dosRS were PCR-amplified from Mabs ATCC 19977 genomic DNA and cloned into the integrative plasmid pMV306, yielding pMV306-dosRS. Alternatively, 220-bp of the promoter region of dosR and the entire coding sequence of the only dosR gene were PCR-amplified and cloned into the integrative plasmid pMV306, yielding pMV306-dosR The sequences of the primers used to generate the different constructs are available upon request.

MIC determinations and drug tolerance assays. The MIC values of DosS inhibitors were determined in cation-adjusted Mueller-Hinton 11 broth and in Synthetic Cystic Fibrosis Medium devoid of mucin and DNA (herein referred to as SCFM), in a total volume of 200 μl in 96-well microtiter plates. Mabs cultures grown to early log phase were diluted to a final concentration of 10⁵ CFU mL⁻¹ and incubated in the presence of serial dilutions of the drugs for 5 d at 37° C. MICs were determined using the resazurin blue test. Drug tolerance assays under hypoxia were conducted in the Wayne model of non-replicating persistence. Cultures of WT Mabs ATCC 19977, the dosRS KO and the complemented mutant strains grown in Dubos-Tween albumin broth to hypoxia (as determined by the decolorization of methylene blue) were added antibiotics and further incubated for 7 d prior to enumeration of surviving bacteria by CFU plating. When DosS inhibitors were used, Mabs cultures were grown to hypoxia as above in the presence of 40 μM of the DosS inhibitors from the time of inoculation; as cultures reached hypoxia, antibiotics and more DosS inhibitors (40 μM) were added and the cultures were left to incubate for 7 d prior to CFU plating.

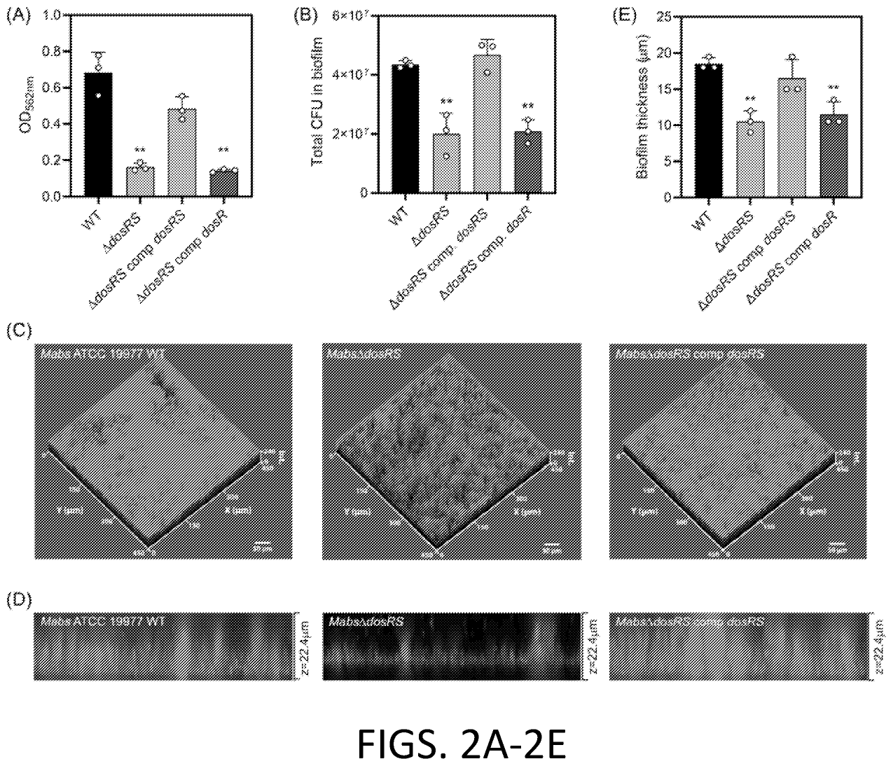

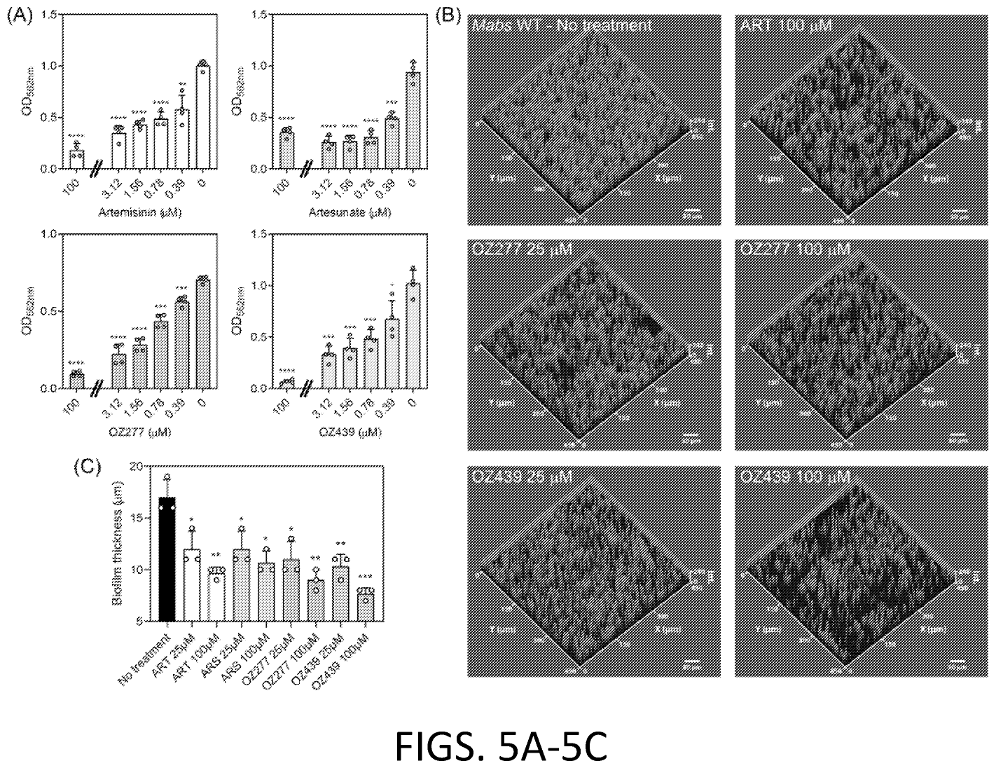

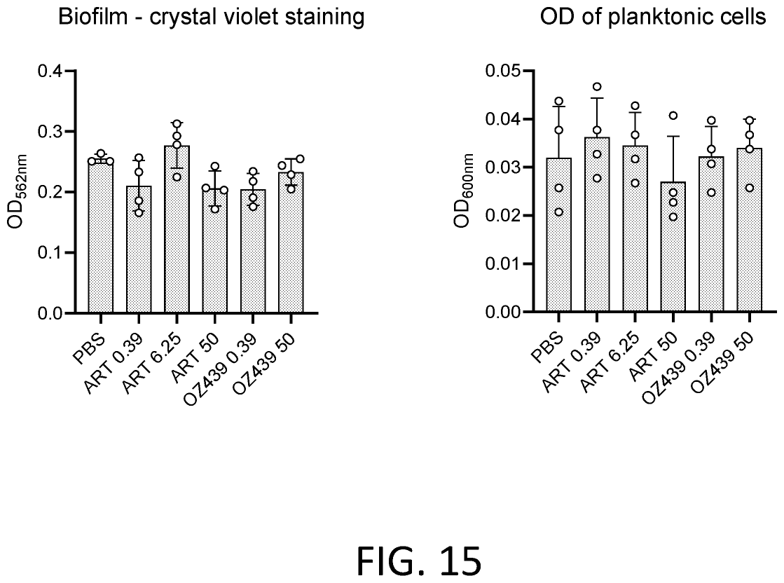

Biofilm assays. MABSC submerged biofilms were formed in 96-well (polystyrene, flat bottom) poly-D-lysine-coated plates in SCFM medium, in the presence or absence of DosS inhibitors. Biofilm formation was monitored by crystal violet staining as follows: Culture medium and planktonic cells were removed from 5-d-old biofilm plate and biofilms were washed gently with PBS prior to adding 100 μL of 0.05% crystal violet solution. After 30 min of incubation at room temperature. the wells were washed with PBS and crystal violet was extracted with 300 μL of 30% acetic acid for 30 min followed by the reading of the absorbance of the solution at 562 nm. The absorbance resulting from the binding of crystal violet to the poly-D-lysine coating was subtracted from all values reported on the graphs presented in FIG. 2A, FIG. 5A, and FIG. 15.

For fluorescence confocal imaging, WT Mabs ATCC 19977. the dosRS KO and the dosRS complemented mutant strains were transformed with pCHERRY3 (Addgene #24659) expressing mCherry. MABSC biofilms were grown for 2 to 6 d on poly-D-lysine-coated μ-Dish^{35 mm, low} (ibidi) at 37° C. in a 5% CO₂ incubator. Biofilms were visualized using a KEYENCE BZ-X700 fluorescence microscope or a ZEISS LSM 510M ETA confocal microscope equipped with a 63×/1.40 plan-Apochromat objective. Two to three independent experiments were performed. Eight to ten different fields were analyzed per strain or treatment condition and images from one representative experiment/field are shown.

Whole cell radiolabeling. Metabolic labeling of Mabs cells with [1,2-¹⁴C]acetic acid (0.5 μCi mL⁻¹; specific activity, 54.3 Ci/mol, PerkinElmer) was performed for 24 hs at 37° C. in Dubos-Tween albumin broth under microaerophilic conditions in standing T25 vented flasks. [1,2-¹⁴C]acetic acid-derived lipids extracted from whole bacterial cells with a mixture of chloroform and methanol (1:2 and 2:1, by vol.) were analyzed by thin-layer chromatograph (TLC) on aluminum-backed silica gel 60-precoated plates F254 (E. Merck) and revealed by Phosphorimaging.

RNA extraction, reverse transcription and RT-qPCR. Two independent cultures of untreated and inhibitor-treated bacteria grown under microaerophilic conditions in Dubos-Tween albumin broth were used for transcriptomics analyses. RNA extraction with the Direct-zol™ RNA Miniprep kit (Zymo Research), reverse transcription reactions using the Superscript IV First-Strand Synthesis System (Thermo Fisher) and RT-qPCRs using the SYBR Green PCR Master Mix (Sigma-Aldrich) were conducted as per the manufacturers' protocols and analyzed on a CFX96 real-time PCR machine (Biorad). PCR conditions: 98° C. (30 s; enzyme activation), followed by 40 cycles of 98° C. (10 s; denaturation) and 60° C. (30 s; annealing/extension). Mock reactions (no reverse transcription) were done on each RNA sample to rule out DNA contamination. The target cDNA was normalized internally to the sigA cDNA levels in the same sample. The following primers were used: MAB_1042c_Fw (5′-ATCGGACTGGAGGCGGATCG-3′ (SEQ ID NO: 1)); MAB_1042c_Rv (5′-AAGAATGGTAGGGCCAGCAC-3′ (SEQ ID NO: 2)); MAB_2489_Fw (5′-CGTCCTTGTCGAGCTATCCA-3′ (SEQ ID NO: 3)); MAB_2489_Rv (5′-CTGCCATCATCCCGGTAAAC-3′ (SEQ ID NO: 4)); MAB_3354_Fw (5′ CCGACCTCGAGCTCCTACAC-3′ (SEQ ID NO: 5)); MAB_3354_Rv (5′-GGATGTAGTCGTGCGGGTTCC-3′ (SEQ ID NO: 6)); MAB_3902c_Fw (5-GCTCACTACGGTCGACCTTA-3′ (SEQ ID NO: 7)); MAB_3902c_Rv (5′-AGGCCATCCCTCCGACCAAA-3′ (SEQ ID NO: 8)); MAB_3903_Fw (5′-CAGACCGCTATCCAACTTGC-3′ (SEQ ID NO: 9)); MAB_3903_Rv (5′-GGTCCAGATACAGGTGCAGA-3′ (SEQ ID NO: 10)); sigA_fwd (5′-CGTTCCTGGACCTGATTCAG-3′ (SEQ ID NO: 11)); and sigA_rev (5′-GTACGTCGAGAACTTGTAACCC-3′ (SEQ ID NO: 12)).

RNA-seq library preparation. RNA was quantified using a Qubit RNA spectrophotometer (Thermo Fisher) and sample quality was assessed using an Agilent High Sensitive RNA Screentape on an Agilent Tapestation, according to the manufacturer's recommendations. All RNA had an RNA Integrity Number (RIN) of greater than 6, indicating sufficient RNA quality for sequencing. Ribosomal RNA depletion was performed using an adapted protocol from Huang et al. ssDNA oligo probes were designed to cover Mabs 16s and 23s rRNA using the RNaseH_depletion scripts (github.com/hym0405/RNaseH_depletion) developed by Huang et al. (The ssDNA oligo probe sequences can be made available upon request.) The oligo probe library was chemically synthesized (Integrated DNA Technologies Inc.), resuspended in a plate format (100 μM), and equimolarly pooled to generate the oligo probe mix used in this study. Ribosomal RNA was depleted from 0.5 μg of total RNA using a 5× probe ratio and 3 μL Hybridase Thermostable RNase H (Lucigen) as recommended by Huang et al. Probes were then removed by DNAse treatment (Thermofisher) followed by a 2× bead clean-up (AMPure RNA, Beckman Counter). Depleted RNA was resuspended in the fragmentation buffer provided with the KAPA RNA Hyperprep kit (Roche). Fragmentation was performed for 6 min at 85° C. followed by the 1^{st} and 2^{nd} strand synthesis. Ligation was performed with 1.5 μM of Kapa Dual-Indexed Adapter (Roche). After the final amplification step (5 cycles), libraries were quantified using Qubit dsDNA BR Assay Kit (Thermo Fisher Sc., USA, MA). and the fragment size was assessed on an Agilent Tapestation using the D1000 Screen tape. Libraries were multiplexed on one sequencing run at equimolar concentrations. Libraries were sequenced using single-end or pair-end reads on an Illumina NextSeq instrument using the mid-output 75 cycles.

RNA-seq data analysis. RNA-seq reads were trimmed for quality score greater than 20 and length greater than 50 using Skewer (version 0.2.2) automatically detecting adapters. Reads were mapped to the Mabs subsp. abscessus ATCC 19977 genome (NC_010397.1) using Bowtie 2 (version 2.3.5) end-to-end alignment with default parameters. Count tables were constructed from sorted BAM files using HTSeq-count (version 0.11.1) set to non-stranded, intersection nonempty using the gff3 file for NC_010397.1 and counting reads on gene_id. Gene expression and differential expression analysis was completed in R (version 3.6.0) using DESeq2 (version 1.26.0). Genes were identified as differentially expressed if they had a log₂ fold change of 1 (Mabs WT untreated vs treated with ART or OZ439) or 2 (Mabs WT vs MabsΔdosRS) and an Benjamini-Hochberg multiple testing correction adjusted p-value of 0.05 or less. The sequencing data described in this publication have been submitted to the NCBI gene expression omnibus (GEO) under BioProject #PRJNA713504 and GEO accession #GSE174310.

UV-visible spectroscopy assay and mass spectrometry. A recombinant form of Mabs ATCC 19977 DosS (MAB_3890c) was produced in E. coli BL21(DE3) using the pET14b expression system (Novagen, Madison, WI) and pGro7, and purified essentially as described. Briefly, His-DosS expression was induced by isopropyl-thio-β-D-galactopyranoside (IPTG, 0.5 mM) at 16° C. for 20 h. The cell pellet was suspended in lysis buffer (50 mM sodium phosphate (pH 7.6), 10% glycerol, 200 mM sodium chloride, 1% Triton X-100, 0.5 mg mL⁻¹ lysozyme, 0.1 mg mL⁻¹ PMSF) and the cell suspension was incubated with shaking at 37° C. for 30 min prior to lysis using a French press (1400-1800 PSI). The soluble extract was applied to a Co²⁺ column (Clontech) and washed with washing buffers (with or without 20 mM imidazole in 50 mM sodium phosphate (pH 7.6), 10% glycerol and 500 mM sodium chloride). The recombinant protein was eluted with 200 mM imidazole in the same buffer. The fractions containing the purified protein were pooled and dialyzed in 20 mM Tris-HCl, pH 7.5. The absorption spectrum of Mabs DosS (5.25 mM) was analyzed as previously described. Briefly, 5.25 μM of purified recombinant DosS protein was deoxygenated in an anaerobic chamber (COY, 95% nitrogen-5% hydrogen). The protein was reduced with 100 μM DTN for 20 min. The reaction was then treated with 100 μM ART, 100 μM MSU-39446, 100 μM OZ439 or an equal volume of DMSO. The UV-visible spectra were recorded for kinetic changes over 2 h using a UV-1800 spectrophotometer (Shimadzu). The experiment was repeated at least twice with similar results.

Mouse infections and treatments. Six to eight-week-old mice were used in all experiments. Fox Chase severe combined immunodeficiency (SCID) Beige mice were ordered from Charles River (North Wilmington, MA, USA).

SCID mice were challenged with the different Mabs WT and recombinant strains using intravenous or infections calibrated to deliver 1.0×10⁶ bacilli per animal. Five animals were infected for each time point. Bacterial inocula were prepared as previously described. At different time points post-infection, bacterial loads in the lungs, liver and spleen were determined. Bacterial counts were determined by plating serial dilutions of organ homogenates on nutrient 7H11-OADC agar (containing 0.4% charcoal in cases where mice received DosS inhibitor and antibiotic treatment) and counting colony-forming units after 5-10 d incubation at 30° C.

OZ277 and OZ439 were formulated in HPMC-SV (0.5% (w/v) hydroxy-propyl methylcellulose, 0.5% (v/v) benzyl alcohol and 0.4% (v/v) Tween 80 in milliQ water).

Tissue distribution of ozonides following in vivo administration. Four to eight Mabs ATCC 19977-infected SCID mice (2 to 4 mice per time point) having received OZ277 and OZ439 as stand-alone drugs by gavage were ethically euthanized on the last day of the experiment for comparison of drug concentrations in the plasma and lung tissues 2 and 24 h (OZ277) or 2 and 48 h (OZ439) after the last dose. For quantitation of OZ277 and OZ439 by HPLC coupled to tandem mass spectrometry (LC-MS/MS), 1 mg mL⁻¹ DMSO stock solutions of OZ277 and OZ439 were serially diluted in 50/50 acetonitrile/water to create standard curves and quality control spiking solutions. Drug-free K2EDTA plasma and lungs from CD-1 mice were obtained from BioIVT for use as blank matrices to build standard curves. Lung tissues were weighed and homogenized in 19 volumes of PBS containing 1% formic acid to 1 part tissue that was pre-chilled. Homogenization was achieved using a FastPrep-24 instrument (MP Biomedicals) and 1.4 mm zirconium oxide beads (Bertin Corp.). PBS was pre-chilled and formic acid was added to stabilize the drugs during homogenization. Spiked matrix standards and quality control (QC) samples were created by adding 10 μL of spiking solutions to 90 μL of drug-free plasma or control lung homogenate. Extraction was performed by adding 200 μL of 1:1 acetonitrile/methanol containing 10 ng mL of the internal standard, verapamil.

LC-MS/MS analysis was performed on a Sciex Qtrap 6500+triple-quadrupole mass spectrometer coupled to a Shimadzu Nexera X2 UHPLC system to quantify each drug in plasma. Chromatography was performed on an Agilent Zorbax SB-C8 column (2.1×30 mm; particle size, 3.5 μm) using a reverse phase gradient elution. Milli-Q deionized water with 0.1% formic acid was used for the aqueous mobile phase and 0.1% formic acid in acetonitrile for the organic mobile phase. Multiple-reaction monitoring (MRM) of precursor/fragment transitions in electrospray positive-ionization mode was used to quantify the analytes. MRM transitions of 470.24/304.00, 393.24/227.00. and 455.40/165.20 were used for OZ439. OZ277, and verapamil respectively. Sample analysis was accepted if the concentrations of the quality control samples were within 20% of the nominal concentration. Data processing was performed using Analyst software (version 1.6.3; Sciex).

Flow cytometry analysis of mCHERRY-expressing MABSC bacilli. WT Mabs ATCC 19977, MabsΔdosRS and MabsΔdosRS complemented with dosRS expressing mCherry from pCHERRY3 (Addgene #24659) were grown under hypoxic conditions in the Wayne model. At the time the cultures reached hypoxia (d 0: i.e., 3 d post-inoculation) or after 3 weeks of incubation under hypoxia (d 21), cultures were centrifuged, and the bacterial pellets were washed three times with 0.85% NaCl containing 0.05% Tween 80. Bacterial cells fixed with 2% paraformaldehyde for 1 h at room temperature and passed through a single cell strainer (40 μm) were then analyzed on a 4-laser Cytek® Aurora flow cytometer using a 561 nm excitation laser and a yellow-green detector module. Freshly-grown Mabs ATCC 19977 harboring or not the mCherry-expressing plasmid were used as positive and negative controls for gating, respectively.

Statistical analysis. The unpaired Student t-test (GraphPad Prism) was used to assess statistical significance between strains and treatment groups in vitro and in vivo.

### 5. Examples

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

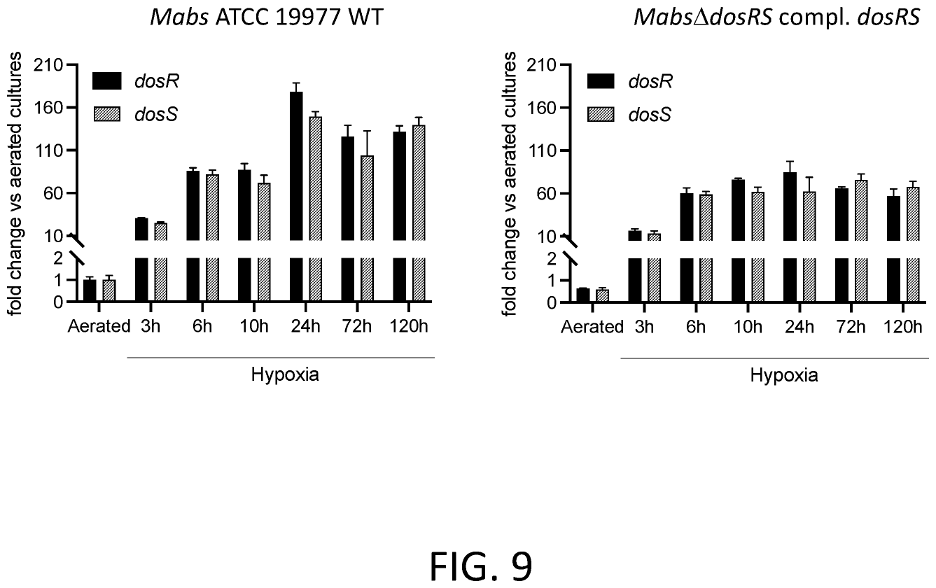

DosRS from MABSC is required for persistence under hypoxia. To determine whether DosRS from MABSC responded to O₂ depletion, the level of expression of dosR and dosS in Mabs ATCC 19977 grown under well-aerated and microaerophilic conditions was compared by RT-qPCR Twenty-four hours after exposure to microaerophilic conditions, when the level of expression of dosR and dosS peaked (FIG. 9), dosR expression was upregulated ˜60-fold relative to cells grown under well-aerated conditions (FIG. 1A).

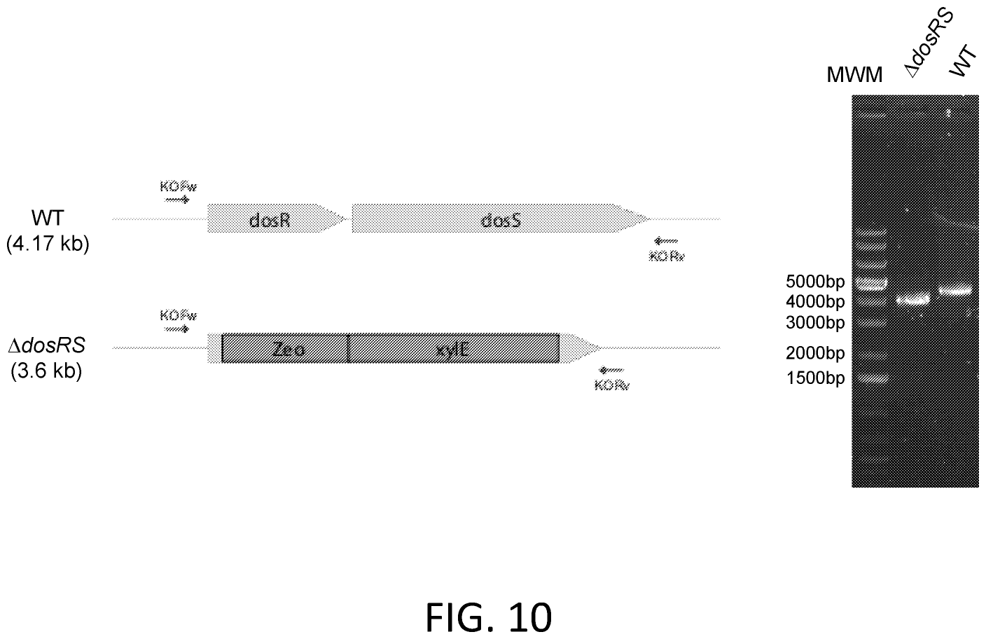

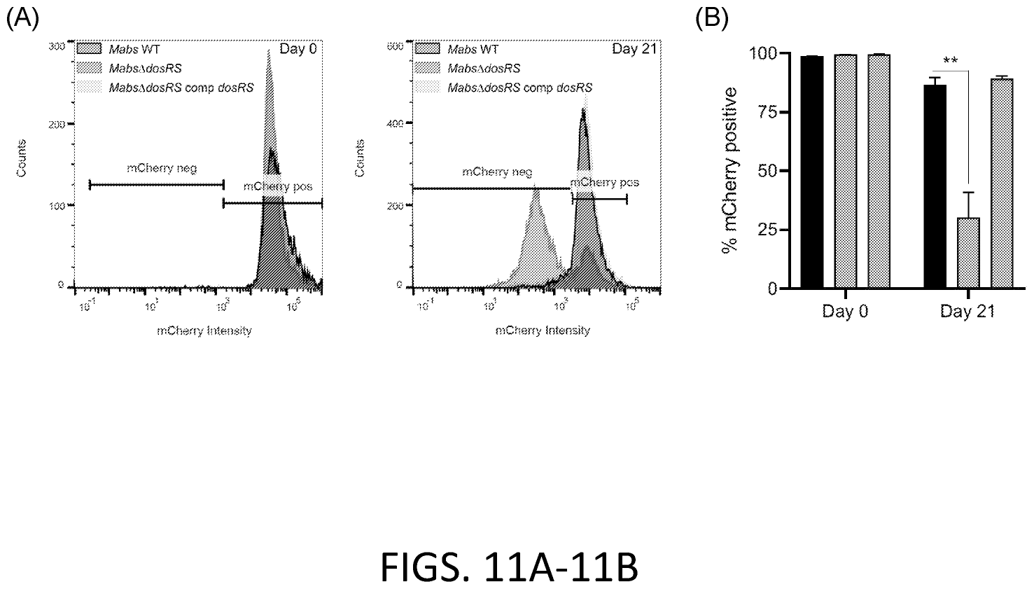

To assess the impact of disrupting the two-component regulator (2CR) on the ability of MABSC to survive under hypoxia, a knock-out (KO) mutant was made of Mabs ATCC 19977 in which the entire dosRS operon was deleted by allelic replacement (FIG. 10). Complemented mutants were generated by transforming the KO mutant, MabsΔdosRS, with integrative plasmids allowing for the expression of the entire dosRS operon or dosR only under control of their own promoter. Relative to the wild-type (WT) parental strain and mutant complemented with dosRS, MabsΔdosRS displayed a clear survival defect in the hypoxic Wayne model of non-replicating persistence characterized by a greater than 1.2 to 1.4 log₁₀ difference in CFUs after 21 d (FIG. 1B). That this decrease in CFU counts reflected loss of viability rather than loss of culturability was verified by monitoring the fluorescence of similarly cultured mCherry-expressing bacterial strains over time. A similar proportion of mCherry-expressing WT, ΔdosRS and dosRS complemented KO bacilli were present in the cultures at the onset of hypoxia, but the percentage of mCherry-expressing (i.e., viable) bacilli after 21 d of hypoxic growth was significantly less (p<0.005) in the case of the mutant compared to the WT and dosRS complemented strains, indicative of the decreased viability of MabsΔdosRS (FIG. 11). Importantly, MabsΔdosRS complemented with only the dosR gene did not survive better than the KO strain (FIG. 1B) indicating that the DosS sensor is required for MABSC to persist under these conditions.

### Example 2

Impact of DosRS on MABSC drug tolerance under hypoxia. DosRST has been shown to be required for Mtb tolerance to antibiotics during hypoxia, a phenotype in large part attributable to the upregulation of the DosR-regulated triglyceride synthase gene, tgsl. To assess the impact of DosRS on the drug tolerance of MABSC under non-replicating hypoxic persistence, Mabs ATCC 19977 WT, MabsΔdosRS. and MabsΔdosRS complemented with dosRS were grown in the same Wayne model as described above and, upon achieving hypoxia, treated for 7 d with antibiotics used in the clinical treatment of MABSC infections, including amikacin (AMK), azithromycin (AZI), imipenem (IMI), and clofazimine (CFZ) prior to enumerating surviving bacteria. The results indicated that while the WT and dosRS complemented mutant were fully tolerant to all antibiotics at the concentrations used herein, MabsΔdosRS displayed a significant (p<0.05) 15, 64 and 52% decrease in survival compared to the DMSO-treated mutant control upon exposure to AMK. AZI and CFZ, respectively (FIG. 1C). Lack of DosRS impaired the ability of MABSC to survive and establish a state of non-replicating, drug-tolerant persistence under hypoxia.

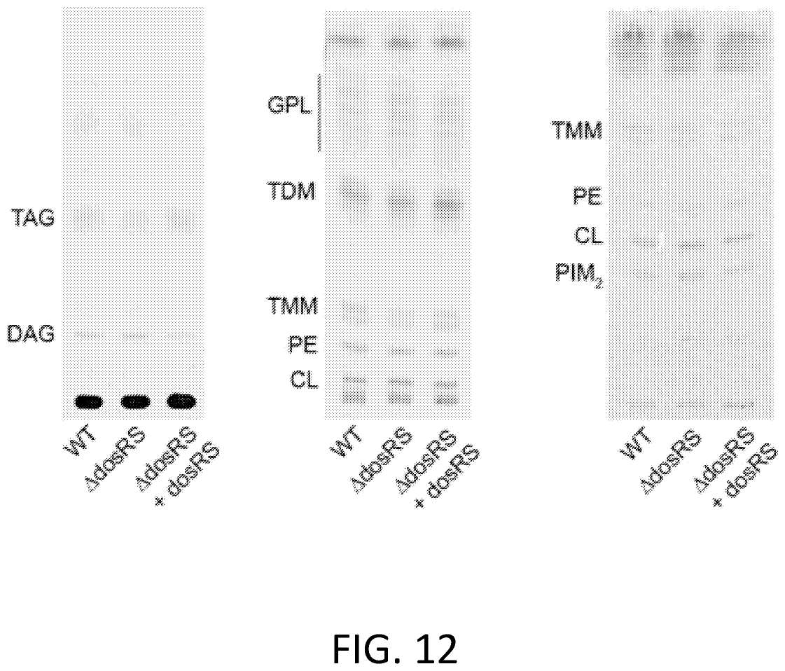

That the drug tolerance of MABSC resulted from the intracellular accumulation of triglycerides following O₂ depletion is unlikely given the result of a metabolic labeling experiment that showed comparable [1,2-¹⁴C]acetate incorporation into these lipids (and other major glycerolipid forms) in the mutant, WT, and dosRS complemented strains (FIG. 12). This finding is in contrast to the situation in Mtb, but it is consistent with the different nature of the DosR regulon in the two species as will be described further.

### Example 3

Effect of DosRS inactivation on MABSC biofilm growth. Reasoning that the disruption of dosRS may impair MABSC response to redox stress and hypoxia and, thus, biofilm development, experiments were conducted to compare the WT and KO strains for their ability to form biofilms in vitro. The biofilm model used to perform this experiment uses a chemically-defined synthetic CF medium (SCFM) to grow MABSC as submerged biofilms in poly-D-lysine-coated plates. SCFM has been shown to closely mimic the nutritional conditions encountered and metabolic adaptation undergone by MABSC in actual CF sputum, making this model more relevant to infection than previously described models based on standard laboratory media.

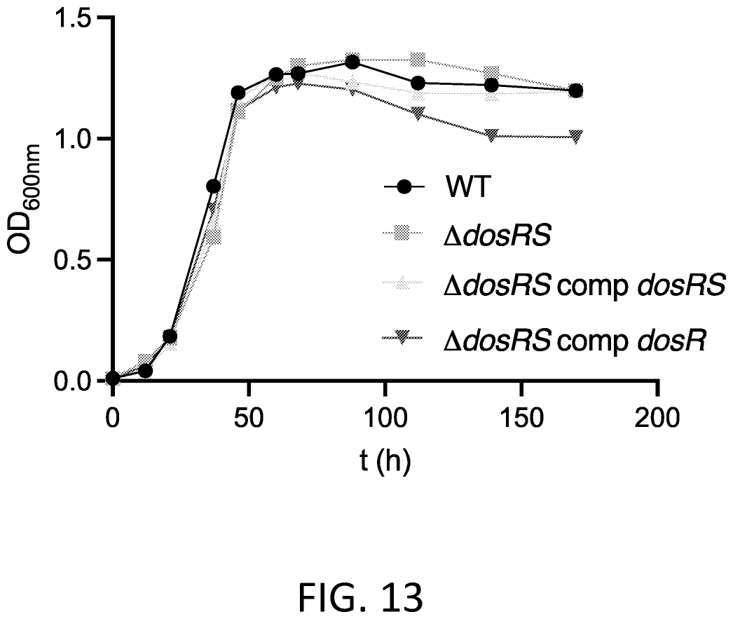

The result from this experiment indicated that despite growing at a similar rate as the WT parent in SCFM under planktonic conditions (FIG. 13), the mutant was ˜4-times less proficient at biofilm formation based on crystal violet staining (FIG. 2A). This defect in biofilm formation also reflected in the number of CFUs associated with WT. mutant, and complemented mutant biofilms (FIG. 2B) and in the confocal fluorescence imaging, including thickness, of biofilms formed by mCherry-expressing WT and dosRS KO strains (FIG. 2). Biofilm formation was largely restored in the dosRS complemented mutant but, again, not in the mutant complemented with dosR only (FIGS. 2A, 2B, 2E).

### Example 4

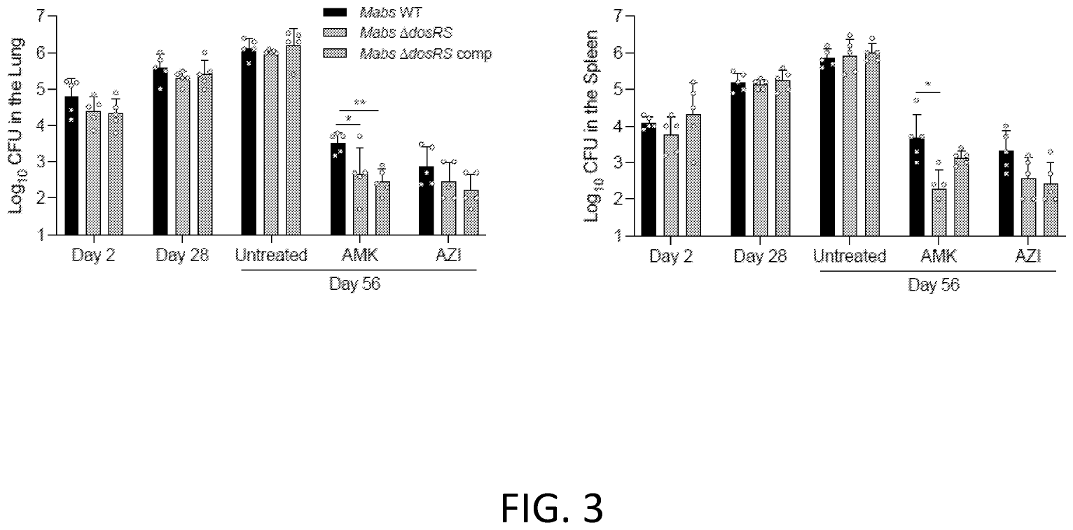

Effect of disrupting dosRS on the susceptibility of MABSC to antibiotics in vivo. The impact of disrupting dosRS on Mtb pathogenesis has proven to be dependent on the animal model used, primarily because DosRS-related phenotypes only clearly express in organized hypoxic lesions. C3HeB/FeJ mice provide such an environment, but their natural resistance to MABSC infection unless continuously treated with corticosteroids limited their potential for the proposed studies. The effect of disrupting dosRS on the virulence and persistence of MABSC in vivo was thus determined in SCID mice due to their permissiveness to MABSC infection and the human-like non-necrotizing and necrotizing granulomas as well as large numbers of foamy cells they develop in which non-replicating MABSC may reside. The WT and mutant strains replicated and persisted similarly in the lungs and spleen of these mice over the course of 56 d (FIG. 3). However, when treated with AMK upon establishment of chronic infection (i.e., as of d 28), significantly (p<0.05) greater bacterial clearance was observed in the case of the mutant compared to the WT parent strain in both organs (FIG. 3). Restoration of WT-like growth in mice infected with the dosRS complemented mutant was visible in the spleen, where the greatest difference between WT and mutant strains was noted, but not in the lungs of AMK-treated animals. The reason for the successful dosRS complementation of the knock-out mutant in all in vitro assays and in the spleen of AMK-treated animals but not in the lung is at present unclear but could be due to apparent differences in the level of expression of dosRS in the complemented strain compared to the WT parent (FIG. 9).

Collectively, these data underscore DosRS as a target of therapeutic interest in MABSC for the potential of inhibitors of this 2CR to impair the ability of MABSC to survive under hypoxic conditions, develop a drug-tolerant persistent state, and form biofilms.

### Example 5

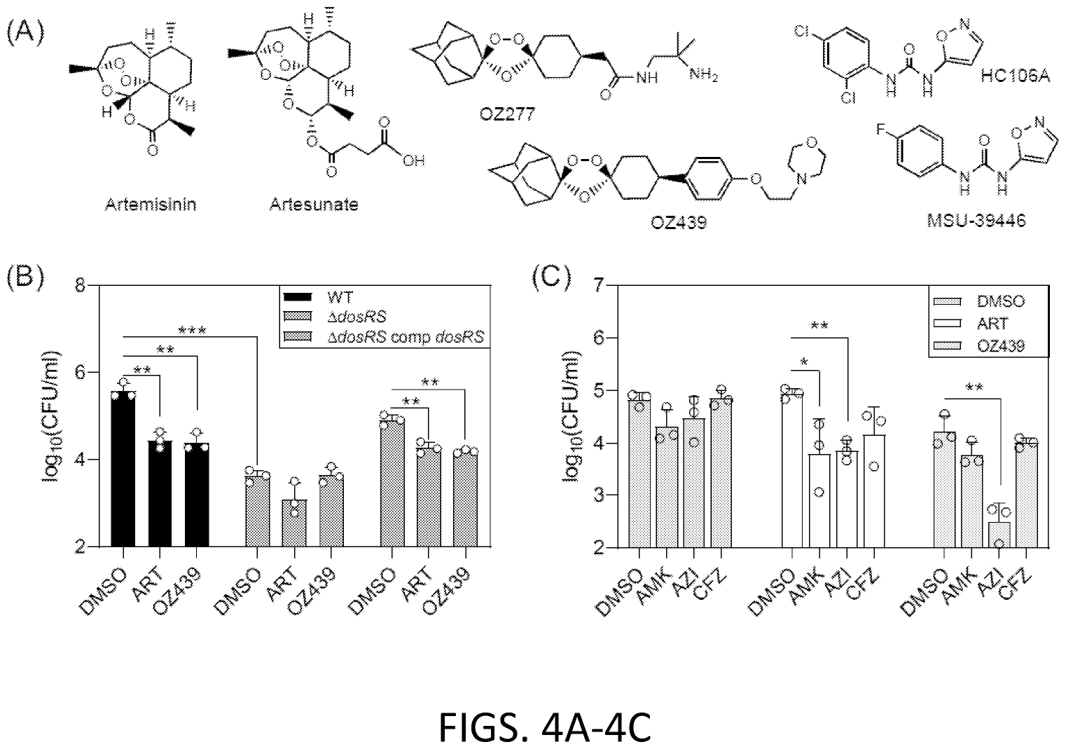

Disruption of MABSC biofilm formation, persistence and drug tolerance under hypoxia by inhibitors of the DosS sensor kinase. A number of inhibitors have been reported to target DosRST signaling in Mtb, including the antimalarial drug artemisinin (ART) and analogs (artesunate [AS], artemether, and dihydroartemisinin), and HC106A (1-(2,4-dichlorophenyl-3-(1,2-oxazol-5-yl)urea) (FIG. 4A). Given the conservation of the DosS proteins from Mtb and MABSC (52% amino acid identity, 68% similarity on a 572 amino acid overlap), experiments were conducted to determine whether Mtb DosS inhibitors were capable of recapitulating in MABSC any of the phenotypic effects associated with the genetic disruption of dosRS. The compounds used in these studies included the optimized HC106A analog, MSU-39446, ART, AS and two synthetic peroxides chosen for their much-improved pharmacokinetic (PK) properties over ART and AS: OZ277 (arterolane; a registered antimalarial drug) and OZ439 (artefenomel; currently in phase 2 clinical trial for the treatment of malaria) (FIG. 4A). All compounds displayed Minimum Inhibitory Concentrations (MICs) against Mabs ATCC 19977 in SCFM and cation-adjusted Mueller-Hinton II media equal to (OZ439) or greater than (all other compounds) 400 μM. Higher concentrations were not tested due to solubility issues.

The effect of ART and OZ439 on the ability of MABSC to persist under hypoxia was first determined by adding 40 μM of each compound to hypoxic cultures of Mabs ATCC 19977 and enumerating surviving bacteria after 7 d of incubation (FIG. 4B). Compared to the DMSO-treated controls, the WT and dosRS complemented strains suffered a significant (p<0.005) decrease in viability upon exposure to either of the compounds (1.12 and 0.64 log₁₀ CFU for ART; 1.18 and 0.72 log₁₀ CFU for OZ439, respectively). MabsΔdosRS-treated cells, in contrast, did not exhibit any significant decrease in viability (beyond that caused by 7 d of hypoxic growth) compared to the DMSO control, indicative of the on-target activity of the compounds.

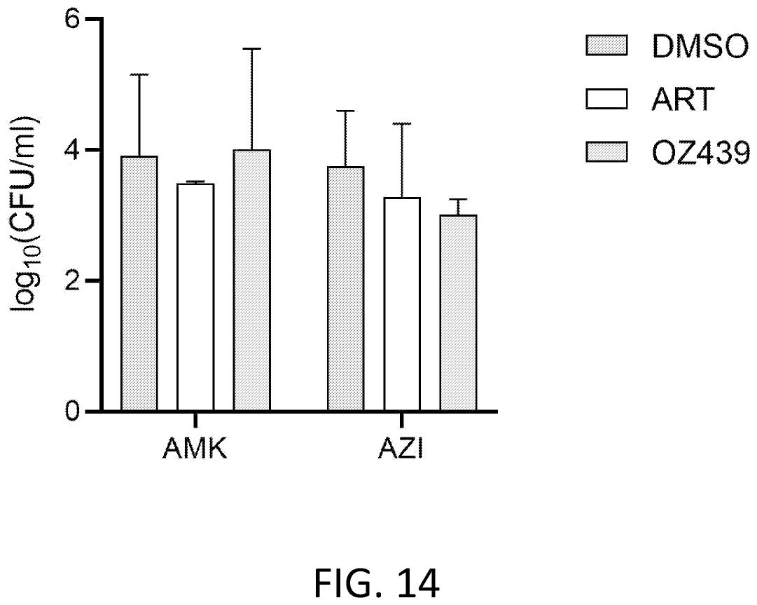

Pretreatment of hypoxic WT Mabs ATCC 19977 cultures with either DMSO, ART or OZ439 for 3 d prior to incubation with AMK, AZI or CFZ for another 7 d further caused a significant (p<0.05) decrease in the tolerance of the ART- and OZ439-treated cells to AZI, and a decrease in the tolerance of the ART-treated cells to AMK, whereas tolerance to CFZ was unaffected by DosS inhibitor treatment (FIG. 4C). In line with the absence of DosS sensor in the dosRS knockout mutant, DosS inhibitor treatment did not significantly enhance the susceptibility of the mutant to AMI or AZI (FIG. 14).

Finally, when tested in the biofilm assay, ART, AS, OZ277, and OZ439 all inhibited biofilm formation with IC₅₀ values (required to inhibit 50% of biofilm formation) of 0.8-1.6 μM (FIGS. 5A-5B). MSU-39446 was the least active with an IC₅₀ value of ˜50 μM. When added to established 4-d old Mabs ATCC 19977 biofilms, none of the compounds dispersed the biofilms under the conditions of the assay, indicating that they were most active in preventing biofilm formation (FIG. 15).

### Example 6

Inhibition of DosRS signaling by artemisinin and synthetic peroxides. To investigate the inhibitory mechanism of ART and OZ439, RNA sequencing-based transcriptional profiling was undertaken on Mabs ATCC 19977 treated with 20 μM of the inhibitors or DMSO as a control under microaerophilic conditions, and the results were compared to the transcriptional profiles of similarly treated MabsΔdosRS cells.

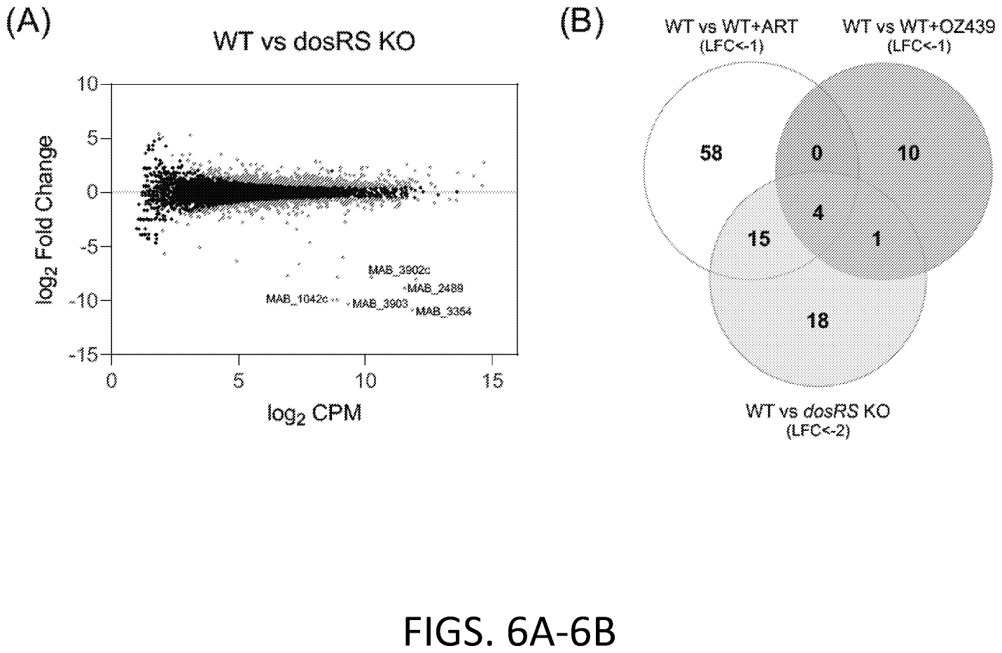

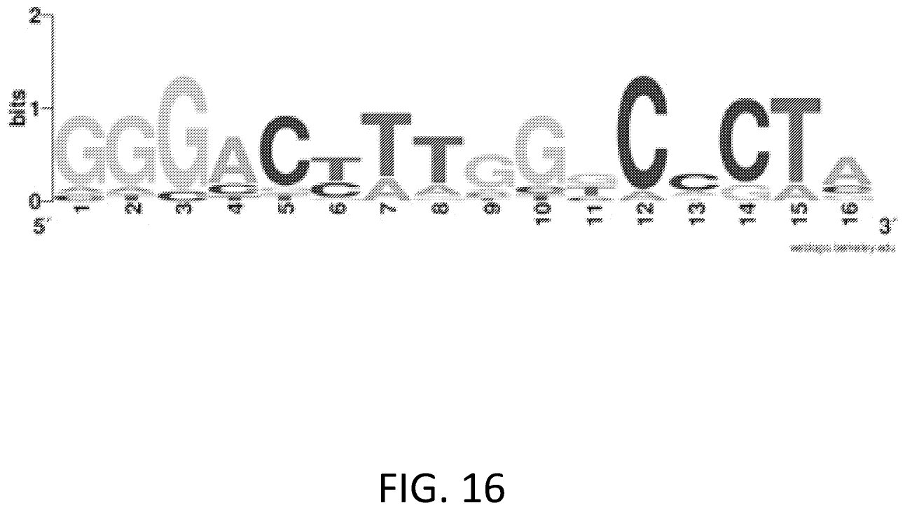

Since the DosR regulon genes of MABSC had not been experimentally characterized, a detailed comparison of the transcriptional profiles of WT Mabs ATCC 19977 and the dosRS KO mutant in the absence of inhibitor treatment was conducted. The list of differentially expressed (DE) genes (log₂ fold change >2 with a false discovery rate adjusted p-value <0.05) was compared to the known DosR regulon of Mtb and the bioinformatically-predicted DosR regulon from MABSC. A total of 38 genes were found to be differentially expressed, 21 of which fall within 7 gene clusters (FIG. 6A: data can be made available upon request). As in other mycobacteria, the DosR regulon of MABSC contains regulators. two universal stress proteins, and a number of genes involved in the maintenance of energy levels and redox potential in the absence of aerobic respiration. The latter include a nitroreductase (MAB_3903) and a nitrate ABC transporter, allowing for the use of nitrate as terminal electron acceptor, a ferredoxin (MAB_2240), the cytochrome c oxidase constituent CtaD (MAB_1042c), the succinate dehydrogenase MAB_2244, and oxidoreductases. Interestingly, the polyphosphate kinase gene ppk2 (MAB_1040) was among the genes expressed at significantly (p<0.05) lower level in the dosRS mutant compared to the WT strain. In Mtb, Ppk2 hydrolyzes polyphosphate, and the accumulation of this important signaling molecule in a ppk2 mutant was shown to impair the ability of Mtb to form biofilms. Among DE genes are MAB_2489 and MAB_3902c. which both have been proposed to be involved in aminoglycoside resistance and might contribute to the observed hyper-susceptibility of MabsΔdosRS to this class of antibiotics in vitro and in vivo (FIG. 6A, FIG. 1C, and FIG. 3). Finally, at least three other genes (MAB_0894c encoding a lipoamide dehydrogenase: MAB_3134c encoding a transcriptional regulator and NO sensor, and MAB_2530c encoding a catalase) are expected to contribute to the intracellular survival of MABSC by protecting the bacilli from host-reactive nitrogen intermediates and reactive oxygen species. A consensus motif was identifiable in the promoter regions of ten of the DE genes or gene clusters of MABSC, which likely represents the binding site of DosR (FIG. 16). Other DE genes that do not appear to be part of the DosR regulon are likely the result of weak binding by DosR or indirect consequences of the inability of MABSC to activate the DosR regulon under microaerophilic conditions.

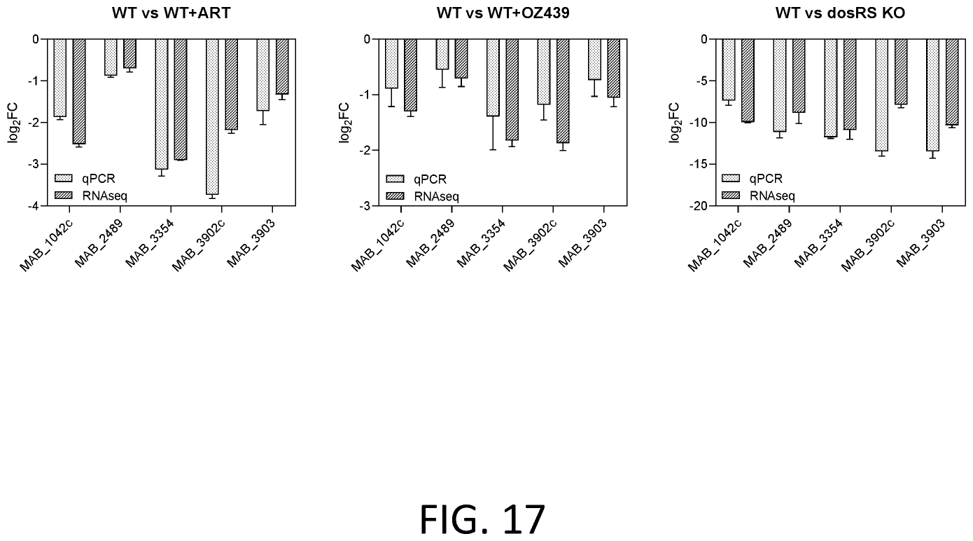

DE genes that changed upon treatment of WT Mabs with ART and OZ439 (log₂ fold change >1 with a false discovery rate adjusted p-value <0.05) showed a striking overlap with the list of DE genes between WT and dosRS KO (log₂ fold change >2 with a false discovery rate adjusted p-value <0.05), even though the magnitude of the changes in expression was less in the inhibitor-treated cells compared to the KO in which DosRS activity is completely abolished (FIG. 6B; data can be made available upon request). ART treatment repressed the expression of half of the genes that are also downregulated in the dosRS KO, whereas OZ439 had a milder effect inhibiting five common genes at the cut-off values set herein. These changes in gene expression indicate that both ART and OZ439 have the ability to prevent the induction of the DosRS regulon under microaerophilic conditions. RT-qPCR confirmed the RNA-seq data for five DosR regulon genes (FIG. 17).

Changes in gene expression in the KO mutant upon exposure to ART and OZ439 were analyzed and compared to the effects of treating the WT strain, with the premise that inhibitors specifically targeting the DosS protein of MABSC will not modulate gene expression in MabsΔdosRS. The mutant treated with ART and OZ439 exhibited only 22 and 10 downregulated genes, respectively, confirming the relative on-target specificity of these compounds.

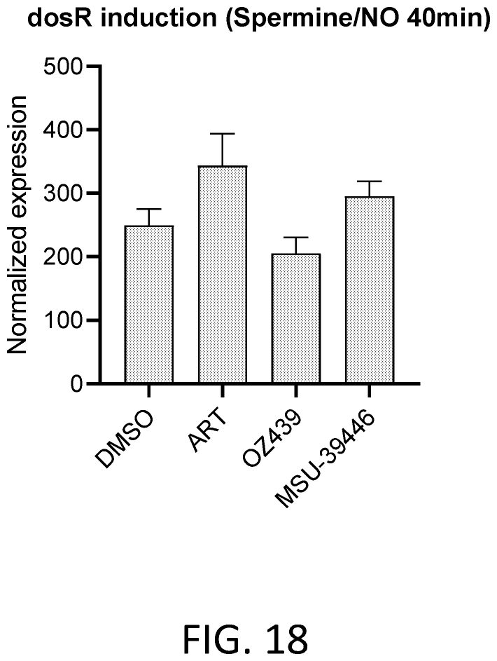

Since the DosRS pathway of MABSC has been reported to be induced by NO, RT-qPCR was used to determine whether ART, OZ439, and MSU-39446 inhibited the induction of DosR-regulated genes in WT Mabs ATCC 19977 following treatment with spermine/nitric oxide (Spermine/NO) for 40 min. Whereas the expression of dosR was induced by more than 240-fold in response to NO, none of the inhibitors tested inhibited NO-signaling (FIG. 18).

### Example 7

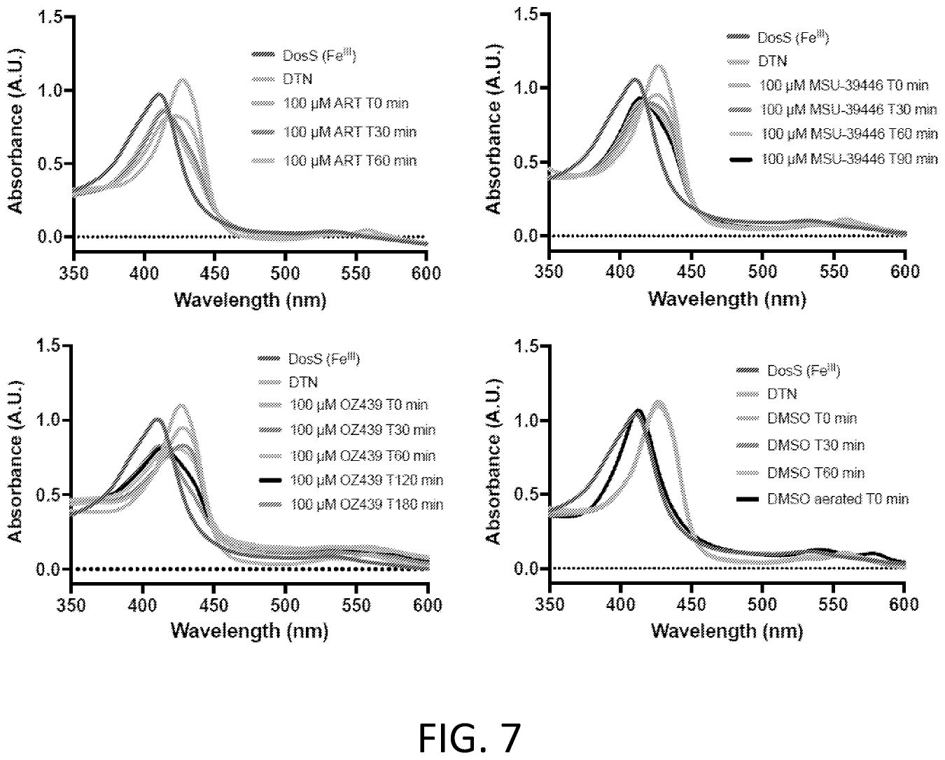

Artemisinin, synthetic peroxides, and MSU-39446 target the sensor kinase heme of MABSC DosS. A UV-visible spectroscopy assay was employed to verify that ART, OZ439, and MSU-39446 inhibited MABSC DosS through modulation of its redox status. To this end, the DosS protein from MABSC was expressed and purified from E. coli as described under Materials and Methods. DosS purified under aerobic conditions has a Soret peak at 410 nm (FIG. 7). Reduction of the heme by dithionite (DTN) shifts the DosS Soret peak to 427 nm. The addition of ART to the reaction mixture caused the Soret peak of DosS to gradually shift back towards the oxidized Soret peak (421 nm at time 0, 414 to 412 nm after 30 or 60 min of incubation). A similar shift towards the oxidized position was also visible upon treatment of purified DosS with OZ439 and MSU-39446. As expected, degassed DMSO did not shift the peak whereas aerated DMSO fully shifted the peak back to the oxidized position (FIG. 7).

### Example 8

Adjunct therapeutic potential of DosS inhibitors in acute and chronic murine models of MABSC infection. Given the ability of DosS inhibitors to decrease the biofilm-forming capacity as well as the survival and drug tolerance of MABSC under hypoxic or microaerophilic conditions, the therapeutic potential of OZ439 and OZ277 was assessed in an in vivo model of MABSC infection, when used alone and in combination with antibiotics. These experiments used similar SCID mouse models as used to compare the virulence and drug tolerance of the WT and dosRS KO mutant (FIG. 3). OZ277 and OZ439 were selected as test compounds for these studies for their safety profile and much improved PK properties over ART.

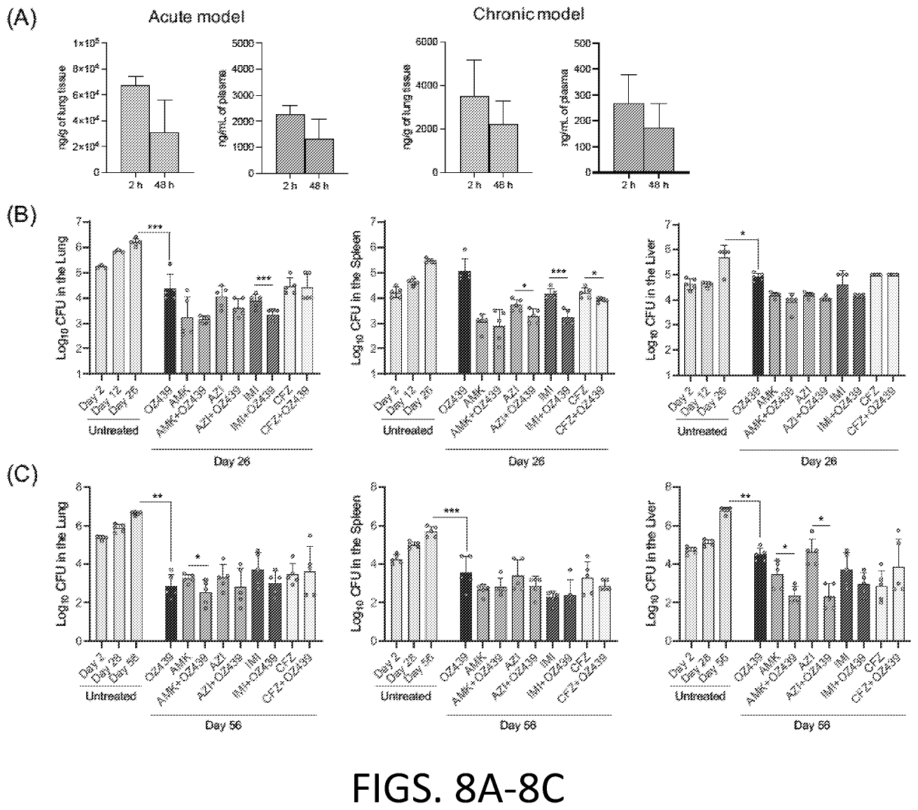

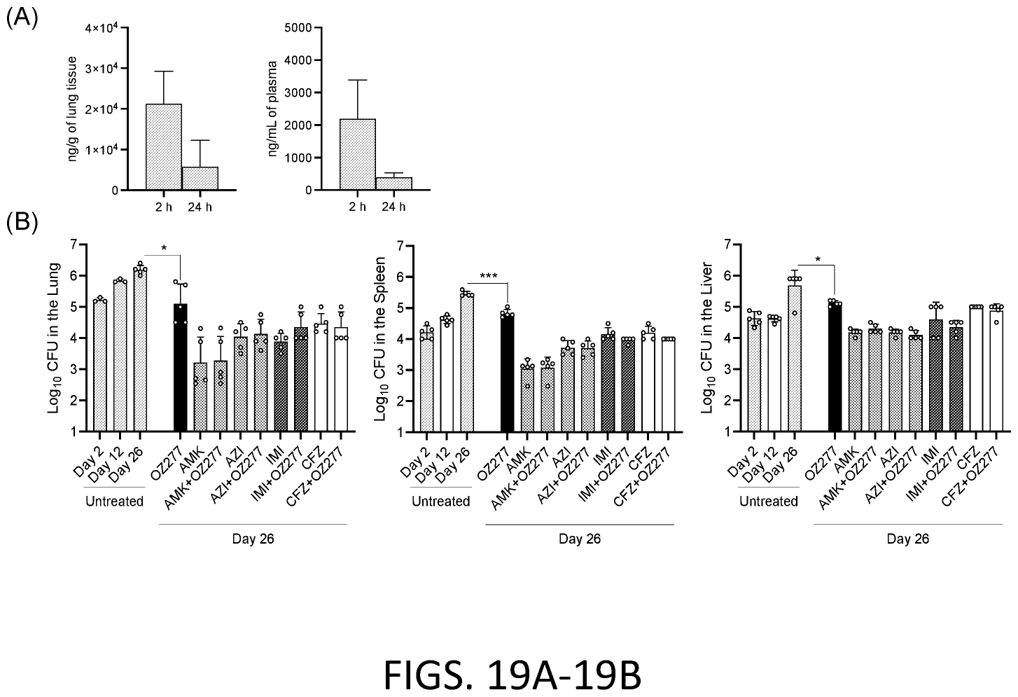

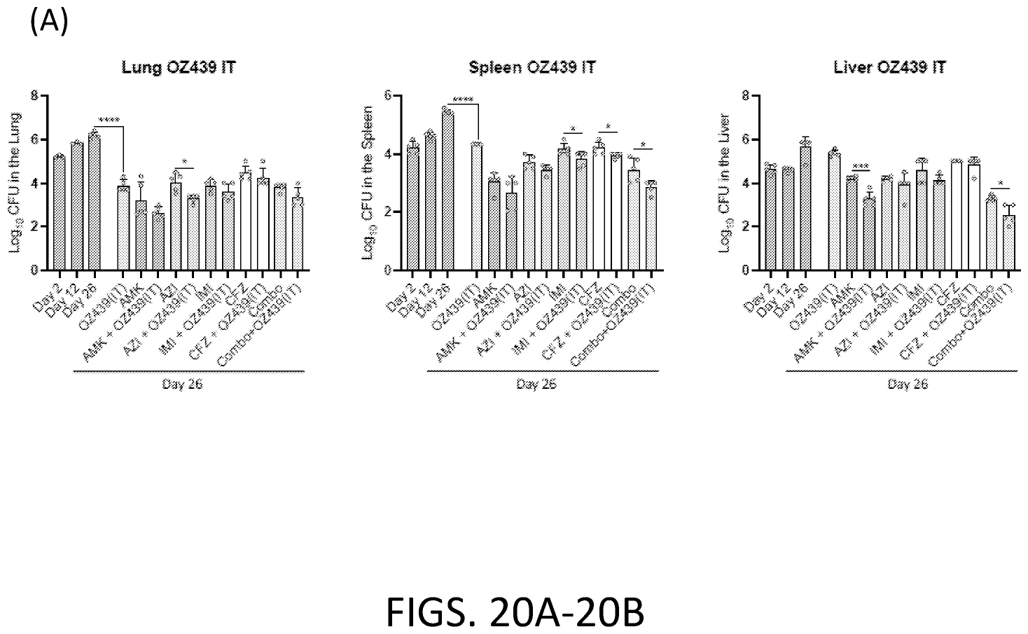

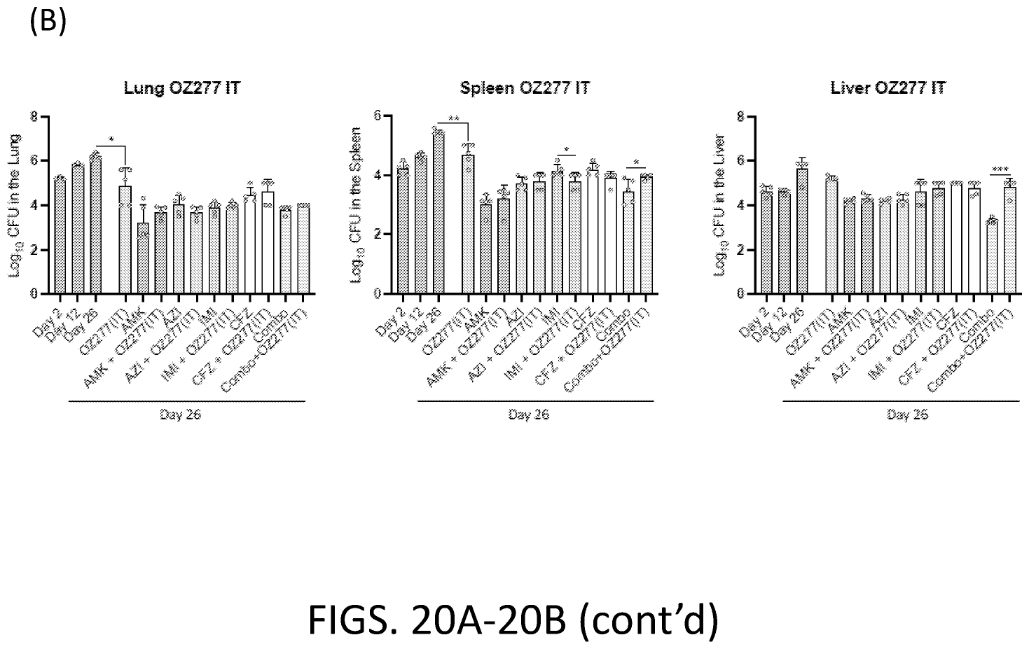

An acute model of MABSC infection was used in an initial experiment in which mice were infected intratracheally with 1.0×10⁶ CFUs of WT Mabs ATCC 19977. On d 12 post-infection during the acute phase of infection, mice were treated daily (7 d a week; every other day for OZ439 owing to the very long half-life of this compound) for 14 days with vehicle used in the formulation of OZ277 and OZ439 (HPMC-SV; see Materials and Methods), OZ439 (200 mg/kg, gavage), OZ277 (200 mg/kg, gavage), AMK (150 mg/kg, subcutaneous injection), AZI (200 mg/kg, gavage), IMI (100 mg/kg, subcutaneous injection), CFZ (20 mg/kg, gavage), and the same four antibiotic treatments in combination with OZ277 or OZ439. Groups of mice were ethically euthanized on d 2, 12 and 26, and processed for bacterial loads in the lungs, liver and spleen. In addition, four MABSC-infected mice that received OZ277 and OZ439 alone by gavage were ethically euthanized on d 26 for comparison of drug concentrations in the plasma and lung tissues at 2, 24, or 48 h after the last dosing. The results indicated good exposure for both compounds with concentrations in the lungs 2, 24, or 48 h after the last oral dosing (FIG. 8A and FIG. 19A) (˜55 to 120 μM for OZ439; ˜10 to 37 μM for OZ277), well above those needed to inhibit biofilm formation in our SCFM model (FIG. 5A). OZ277 and OZ439 by themselves significantly reduced bacterial burdens in all organs of infected mice (p<0.05), with the exception of OZ439 in the spleen (FIG. 8B and FIG. 19B. Remarkably, the reduction in bacterial load in the lung following OZ439 treatment for 14 d (˜1.8 log₁₀) was of similar magnitude as that observed upon treatment with CFZ and IMI (FIG. 8B). When used in combination with antibiotics, OZ439 further potentiated the activity of AZI, IMI and CFZ in the spleen and/or lung, resulting in significantly (p<0.05) greater reduction in bacterial loads in these organs than seen in infected animals treated with antibiotics alone (FIG. 8B).

In a follow-up experiment, the therapeutic efficacy of OZ439 at the reduced dose of 50 mg/kg, alone or in combination with the same four antibiotics, was tested in a chronic SCID mouse model of MABSC infection. Mice were infected intravenously with 1.0×10⁶ CFUs of WT Mabs ATCC 19977. On d 28 post-infection, upon establishment of chronic infection, mice were treated daily (7 ds a week; every other day for OZ439) for 28 d with formulation vehicle, OZ439 (50 mg/kg, gavage), AMK, AZI, IMI and CFZ (same dosing as in the previous experiment) and the same four antibiotics in combination with OZ439. Determination of OZ439 exposure in this model indicated concentrations in the lungs 2 and 48 h after the last oral dose in the range of 6.2 and 3.9 μM, respectively, still above those needed to inhibit biofilm formation in SCFM and considerably below the MIC value of OZ439 in laboratory medium (400 μM) (FIG. 8A). Analysis of bacterial loads in the lungs, liver, and spleen of the different treatment groups on d 2, 28 and 56 indicated that the efficacy of OZ439 by itself was comparable to that of all four antibiotics in the chronic model suggestive of the greater activity of the DosS inhibitor during the chronic vs. acute phase of infection (FIGS. 8B-8C). When used in combination with antibiotics. OZ439 potentiated the activity of AZI in the liver and that of AMK in the lung and liver.

### Example 9

Mechanisms underlying the therapeutic benefit of OZ439 against NTM infections. The concentrations of OZ277 and OZ439 that killed MABSC in vivo were at least two orders of magnitude lower than their in vitro MIC. Also, DosRS is a 2CR involved in the regulation of >38 genes in response to stresses encountered in a variety of microenvironments within the infected host (e.g., inside phagocytic cells, within granulomas and in biofilms). Thus, the mechanism underlying the therapeutic and adjunct therapeutic efficacy of DosS inhibitors in vivo is likely to be multifactorial and to impact MABSC bacilli residing both intra- and extra-cellularly. Additionally, the dramatic decrease in survival of DosS inhibitor-treated WT Mabs in SCID mice while the dosRS KO mutant failed to show any virulence attenuation in the same mouse model, could indicate that synthetic peroxides exert at least part of their activity through the modulation of host responses rather than predominantly through the inhibition of DosS. Indeed, artemisinin-based compounds and synthetic peroxides (including OZ277 and OZ439) are known to display a broad-spectrum of antimicrobial and anticancer activities, some of which were attributed to the ability of these compounds to enhance oxidative stress, apoptosis, autophagy, and lysosomal acidification. The fact that synthetic peroxides significantly reduced MABSC bacillary loads in infected SCID mice that fail to mount normal adaptive immune responses further suggests that host-directed effects involving innate immune responses in particular may contribute to the efficacy of these drugs. Of note, there is growing evidence indicating that autophagy-activating agents may be beneficial in restricting intracellular growth of mycobacteria, including MABSC.

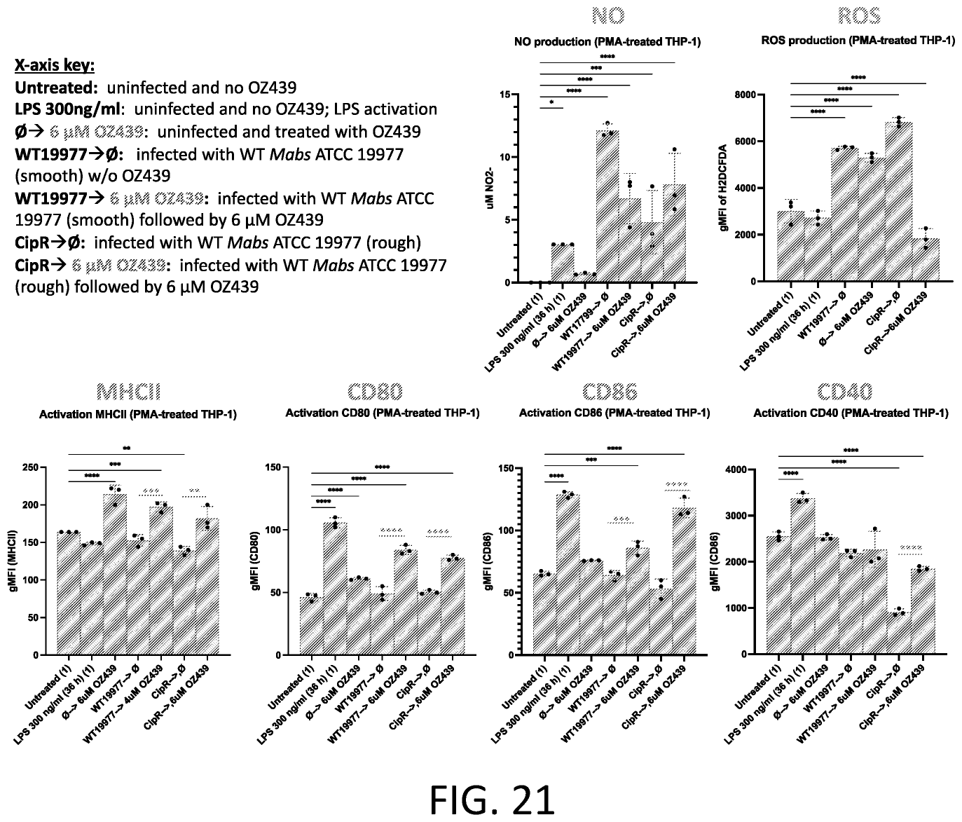

Therefore, experiments were conducted to assess host-directed effects of OZ439 on infected and non-infected THP-1 cells, as illustrated in FIG. 21. THP-1 cells were plated at 1×10⁶ cells per ml in 24-well plates and treated for 24 hrs. with 10 ng/ml PMA for differentiating to macrophages. PMA medium was removed, and cells were cultured for an additional 24 hrs. Cells were then treated with either nothing, 300 ng/ml LPS or either Mabs ATCC19977 (smooth) or Mabs ATCC19977 (rough) (aka CipR) at an MOI of 5. After 9 h, the medium was removed and replaced with medium containing either nothing or 6 μM of OZ439 in triplicate wells and THP-1 cells were cultured for an additional 30 hr. Supernatants were harvested and assayed for NO production by the Griess Assay. Half the cells were stained with H2DCFDA for ROS analysis and half were stained for macrophage activation markers and analyzed by flow cytometry. Statistical differences between treatment groups were determined by one way ANOVA with Dunnett's multiple comparison test (*=p<0.05; ***=p<0.001; ****=p<0.0001).

Infection of THP-1 cells with the rough and smooth Mabs ATCC19977 strains alone stimulated increases in NO production with significantly greater increases for the smooth morphotype strain. Addition of 6 μM OZ439 to the medium reverted this activation in the case of the Mabs ATCC19977 smooth strain. ROS was upregulated in the OZ439-treated cultures in the absence of Mabs (rough or smooth) infection. However, overall, ROS production was decreased in OZ439-treated cultures that were infected with Mabs and significantly more so in cells infected with the rough morphotype Mabs compared to the smooth morphotype. Treatment of the cells with 6 μM OZ439 alone showed increased expression of some macrophage activation markers (MHC class II and CD80). Interestingly, infection of THP-1 cells with Mabs followed by OZ439 treatment showed significant increases in all macrophage activation markers (MHC class 11, CD80, CD86, CD40) when compared to those that were not treated with OZ439 (see red asterisks) (FIG. 21).

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A composition for treating a non-tuberculous mycobacterial infection in a subject comprising: a therapeutically effective dose of at least one anti-malarial agent, and a pharmaceutically acceptable carrier.

2. The composition of claim 1, wherein the non-tuberculous mycobacterial infection is caused by a non-tuberculous mycobacteria (NTM) selected from the group consisting of: M. abscessus subsp. abscessus, M. abscessus subsp. massiliense, M. abscessus subsp. bolletii, Mycobacterium avium complex, M. kansasii, M. chelonae, M. fortuitum, and M. xenopi, M. peregrinum, M. mucogenicum, M. senegalense, M. immunogenum, M. simiae, M. gordonae, M. septicum, M. malmoense, M. goodii, M. haemophilum, M. genavense, M. scrofulaceum, M. lentiflavum, M. bohemicum, and M. marinum.

3. The composition of claim 1, wherein the non-tuberculous mycobacterial infection is caused by a mycobacterium selected from the group consisting of: M. leprae, and M. ulcerans

4. The composition of any one of claims 1 to 3, wherein the composition treats and/or prevents at least one symptom of the non-tuberculous mycobacterial infection in the subject.

5. The composition of claim 4, wherein the symptom is selected from the group consisting of: a cough, weight loss, appetite loss, fatigue, shortness of breath, a fever, and night sweats.

6. The composition of any one of claims 1 to 5, wherein the at least one anti-malarial agent comprises a trioxolane (secondary ozonide).

7. The composition of any one of claims 1 to 5, wherein the at least one anti-malarial agent comprises a synthetic peroxide.

8. The composition of any one of claims 1 to 7, wherein the at least one anti-malarial agent comprises arterolane (OZ277).

9. The composition of any one of claims 1 to 7, wherein the at least one anti-malarial agent consists essentially of arterolane (OZ277).

10. The composition of any one of claims 1 to 7, wherein the at least one anti-malarial agent comprises ozonide OZ439.

11. The composition of any one of claims 1 to 7, wherein the at least one anti-malarial agent consists essentially of ozonide OZ439.

12. The composition of any one of claims 1 to 7, wherein the at least one anti-malarial agent comprises OZ277 and OZ439.

13. The composition of any one of claims 1 to 7, wherein the at least one anti-malarial agent consists essentially of OZ277 and OZ439.

14. The composition of any one of claims 1 to 13, wherein the at least one anti-malarial agent is present in the composition in an amount ranging from about 5 mg to about 1200 mg.

15. The composition of claim 8, wherein the OZ277 is present in the composition in an amount ranging from about 5 mg to about 1200 mg.

16. The composition of claim 11, wherein the OZ439 is present in the composition in an amount ranging from about 5 mg to about 1200 mg.

17. The composition of any one of claims 1 to 16, wherein composition further comprises at least one antibiotic.

18. The composition of claim 17, wherein the at least one antibiotic is selected from the group consisting of: clarithromycin, azithromycin, rifampin, rifabutin, ethambutol, streptomycin, amikacin, ciprofloxacin, doxycycline, clofazimine, isoniazid, linezolid, moxifloxacin, trimethoprim/sulfamethoxazole, cefoxitin, imipenem, and tigecycline.

19. The composition of any one of claims 1 to 16, wherein composition does not comprise an antibiotic.

20. A method of treating and/or preventing a non-tuberculous mycobacterial infection in a subject comprising administering the composition of any one of claims 1 to 19 to the subject.

21. The method of claim 20, wherein the composition is administered in a single dose.

22. The method of claim 20, wherein the composition is administered in multiple doses.

23. The method of claim any one of claims 20 to 22, wherein the composition is administered daily, weekly, bi-weekly or monthly.

24. The method of claim any one of claims 20 to 23, wherein the composition is formulated as a tablet, capsule, powder, granule, sachet, gel, liquid, spray, suspension, or inhalant.

25. The method of any one of claims 20 to 24, wherein the composition is administered to the subject orally, sublingually, topically, intravenously, subcutaneously, transcutaneously, nasally, vaginally, rectally, or by inhalation.

26. The method of any one of claims 20 to 25, wherein the composition is administered to the subject at a dose ranging from about 50 mg/kg to about 1200 mg/kg.

27. The method of any one of claims 20 to 25, wherein the composition is administered to the subject in an amount ranging from about 50 mg/dose to about 1200 mg/dose.

28. The method of any one of claims 20 to 27, wherein the composition is administered with an antibiotic.

29. The method of claim 28, wherein the composition is administered with the antibiotic in the same dose or in a different dose.

30. The method of any one of claims 20 to 27, wherein the composition is not administered with an antibiotic.

31. The method of any one of claims 20 to 30, wherein administration of the composition enhances at least one of oxidative stress, apoptosis, autophagy and/or lysosomal acidification in the subject.

32. The method of any one of claims 20 to 30, wherein administration of the composition reduces and/or prevents biofilm formation in the subject.


SEQUENCE LISTING (12 sequences)

SEQ ID NO: 1 - DNA, 20 bp, Artificial sequence
  Synthetic
atcggactggaggcggatcg

SEQ ID NO: 2 - DNA, 20 bp, Artificial sequence
  Synthetic
aagaatggtagggccagcac

SEQ ID NO: 3 - DNA, 20 bp, Artificial sequence
  Synthetic
cgtccttgtcgagctatcca

SEQ ID NO: 4 - DNA, 20 bp, Artificial sequence
  Synthetic
ctgccatcatcccggtaaac

SEQ ID NO: 5 - DNA, 20 bp, Artificial sequence
  Synthetic
ccgacctcgagctcctacac

SEQ ID NO: 6 - DNA, 21 bp, Artificial sequence
  Synthetic
ggatgtagtcgtgcgggttcc

SEQ ID NO: 7 - DNA, 20 bp, Artificial sequence
  Synthetic
gctcactacggtcgacctta

SEQ ID NO: 8 - DNA, 20 bp, Artificial sequence
  Synthetic
aggccatccctccgaccaaa

SEQ ID NO: 9 - DNA, 20 bp, Artificial sequence
  Synthetic
cagaccgctatccaacttgc

SEQ ID NO: 10 - DNA, 20 bp, Artificial sequence
  Synthetic
ggtccagatacaggtgcaga

SEQ ID NO: 11 - DNA, 20 bp, Artificial sequence
  Synthetic
cgttcctggacctgattcag

SEQ ID NO: 12 - DNA, 22 bp, Artificial sequence
  Synthetic
gtacgtcgagaacttgtaaccc